(19) United States
(12) Patent Application Publication
(10) Pub. No.: US 2024/0158479 A1
(43) Pub. Date: May 16, 2024

(54) NEUTRALIZING ANTI-SARS-COV-2 ANTIBODIES, CHIMERIC IMMUNOGENS, AND METHODS OF USE THEREOF

(71) Applicant: Rutgers, The State University of New Jersey, New Brunswick, NJ (US)

(72) Inventors: Alok K. Choudhary, Newark, NJ (US); Abraham Pinter, Lakewood, NJ (US)

(73) Assignee: Rutgers, The State University of New Jersey, New Brunswick, NJ (US)

(21) Appl. No.: 18/504,832

(22) Filed: Nov. 8, 2023

Related U.S. Application Data
(60) Provisional application No. 63/487,164, filed on Feb. 27, 2023.
(60) Provisional application No. 63/382,752, filed on Nov. 8, 2022.

(51) Int. Cl. C07K 16/10; A61P 31/14

(52) U.S. Cl. CPC C07K 16/1003; A61P 31/14; A61K 2039/507

(57) ABSTRACT

This disclosure provides novel potent neutralizing antibodies directed against SARS-CoV-2 Omicron and other variants, related nucleic acids, related cells, related kits, related compositions, and related methods or uses. Also provided in this disclosure are novel combinations of anti-SARS-CoV-2 antibodies that exhibit synergistic effects in neutralizing SARS-CoV-2 variants. Furthermore, this disclosure provides novel chimeric immunogens for inducing effective immune responses against SARS-CoV-2 infections.

### CROSS-REFERENCE TO RELATED APPLICATIONS

This application claims priority under 35 U.S.C. § 119(e) to U.S. Provisional Patent Application No. 63/487,164, filed Feb. 27, 2023, and U.S. Provisional Patent Application No. 63/382,752, filed Nov. 8, 2022. The foregoing applications are incorporated by reference herein in their entireties.

### STATEMENT REGARDING FEDERALLY SPONSORED RESEARCH

This invention was made with government support under grant number AI152157 awarded by the National Institutes of Health. The government has certain rights in the invention.

### FIELD OF THE INVENTION

The present invention relates to antibodies directed to epitopes of SARS-CoV-2 Coronavirus 2 Omicron and other CoV-2 variants (“SARS-CoV-2 variants”). The present invention further relates to the preparation and use of neutralizing antibodies directed to the SARS-CoV-2 spike (S) glycoproteins for the prevention and treatment of SARS-CoV-2 infection.

### BACKGROUND

Severe Acute Respiratory Syndrome Coronavirus (SARS-CoV-2) infections can cause serious diseases in susceptible patients, including the elderly and those who are immunosuppressed and/or have underlying medical conditions. An important and effective treatment for such individuals is immunotherapy with selected monoclonal antibodies (mAbs) that possess potent neutralizing activity against infecting strains. For antibody therapies to be successful, they must retain potent neutralizing breadth against the emerging SARS-CoV-2 lineages that carry mutations. Several mAb combinations were highly effective against the initial CoV-2 pathogen and early variants and received emergency use authorization, but they lost effectiveness against the emerging escape mutants. Currently, none of the antibody-based COVID-19 immunotherapeutic remains effective currently circulating, more transmissible, and highly mutated Omicron subvariants (e.g., BQ.1.1, and XBB.1.5), and pose a threat to vulnerable populations who rely on antibody-based therapeutics. These highlight the need for more effective monoclonal antibodies that can be used to treat current and future infections and improved vaccines for sterilizing immunity.

### SUMMARY

In one aspect, this disclosure provides a novel isolated and recombinantly modified anti-SARS-CoV-2 neutralizing antibody or antigen-binding fragment thereof that binds specifically to an antigen of SARS-CoV-2 Coronavirus 2 Omicron and other variants. In some embodiments, the SARS-CoV-2 antigen comprises a spike (S) polypeptide, such as a S polypeptide of a human or an animal SARS-CoV-2. In some embodiments, the SARS-CoV-2 antigen comprises the receptor binding domain (RBD) (e.g., residues 319-541 of the S polypeptide).

In some embodiments, the antibody or antigen-binding fragment thereof is capable of neutralizing a plurality of SARS-CoV-2 strains.

In some embodiments, the antibody or antigen-binding fragment thereof comprises: three heavy chain complementarity determining regions (HCDRs) (HCDR1, HCDR2, and HCDR3) of a heavy chain variable region (HCVR) having the amino acid sequence of SEQ ID NO: 7; and three light chain CDRs (LCDR1, LCDR2, and LCDR3) of a light chain variable region (LCVR) having the amino acid sequence of SEQ ID NO: 8.

In some embodiments, HCDR1, HCDR2, HCDR3, LCDR1, LCDR2, and LCDR3 comprise respective amino acid sequences of SEQ ID NOs: 1-6.

In some embodiments, the antibody or antigen-binding fragment thereof comprises a HCVR having the amino acid sequence of SEQ ID NO: 7. In some embodiments, the antibody or antigen-binding fragment thereof comprises a LCVR having the amino acid sequence of SEQ ID NO: 8. In some embodiments, the antibody or antigen-binding fragment thereof comprises a HCVR having the amino acid sequence of SEQ ID NO: 7, and a LCVR having the amino acid sequence of SEQ ID NO: 8.

The antibody or antigen-binding fragment thereof comprises a heavy chain having the amino acid sequence of SEQ ID NO: 9. In some embodiments, the antibody or antigen-binding fragment thereof comprises a light chain having the amino acid sequence of SEQ ID NO: 10. In some embodiments, the antibody or antigen-binding fragment thereof comprises a heavy chain having the amino acid sequence of SEQ ID NO: 9, and a light chain having the amino acid sequence of SEQ ID NO: 10.

In some embodiments, the antibody or antigen-binding fragment thereof is ACOVIMAB.

In some embodiments, the antibody or antigen-binding fragment thereof is a multivalent antibody that comprises a first target binding site that specifically binds to an epitope within the S polypeptide, and a second target binding site that binds to a different epitope on the S polypeptide or a different molecule. In some embodiments, the multivalent antibody is a bivalent or bispecific antibody.

In some embodiments, the antibody is a monoclonal antibody. In some embodiments, the antibody is a chimeric antibody, a humanized antibody, or a humanized monoclonal antibody. In some embodiments, the antibody is a single-chain antibody, Fab or Fab2 fragment.

In some embodiments, the antibody comprises M428L/N434S substitutions in the Fc region that confers enhanced binding to a neonatal Fc receptor.

In another aspect, this disclosure further provides a nucleic acid molecule comprising a polynucleotide sequence encoding the antibody or antigen-binding fragment thereof described herein, a vector comprising the nucleic acid molecule described herein, and a host cell comprising the nucleic acid molecule or the vector, as described herein.

In another aspect, this disclosure further provides a method of preparing the antibody or antigen-binding portion thereof. In some embodiments, the method comprises: culturing the host cell, as described herein, in a medium under conditions permitting expression of a polypeptide encoded by the nucleic acid molecule or the vector and assembling of an antibody or fragment thereof; and isolating the antibody or fragment from the cultured cell or the medium of the cell.

In another aspect, this disclosure also provides a pharmaceutical composition comprising the antibody or antigen-binding fragment thereof, the nucleic acid molecule, or the vector, as described herein; and optionally a pharmaceutically acceptable carrier or excipient.

In another aspect, this disclosure additionally provides a kit comprising a pharmaceutically acceptable dose unit of the antibody or antigen-binding fragment thereof or the pharmaceutical composition, as described herein.

Also within the scope of this disclosure is use of the antibody or antigen-binding fragment thereof or the pharmaceutical composition, as described herein, in the manufacture of a medicament for the diagnosis, prophylaxis, treatment, or combination thereof of a condition resulting from a SARS-CoV-2.

In yet another aspect, this disclosure provides a method of neutralizing SARS-CoV-2 or treating a SARS-CoV-2 infection in a subject. In some embodiments, the method comprises administering to a subject in need thereof a therapeutically effective amount of the antibody or antigen-binding fragment thereof or a therapeutically effective amount of the pharmaceutical composition, as described herein.

In some embodiments, the antibody or antigen-binding fragment thereof is administered to the subject intravenously, subcutaneously, or intraperitoneally.

In some embodiments, the method further comprises administering to the subject a therapeutically effective amount of a second therapeutic agent or therapy. In some embodiments, the second therapeutic agent comprises an anti-inflammatory drug or an antiviral compound. In some embodiments, the antiviral compound may include: acyclovir, gancyclovir, vidarabine, foscarnet, cidofovir, amantadine, ribavirin, trifluorothymidine, zidovudine, didanosine, zalcitabine, or interferon. In some embodiments, the interferon is an interferon-α or an interferon-β.

In another aspect, this disclosure also provides a method for detecting the presence of SARS-CoV-2 in a sample. In some embodiments, the method comprises: contacting a sample with the antibody or antigen-binding fragment thereof described herein; and determining binding of the antibody or antigen-binding fragment to one or more SARS CoV-2 antigens, wherein binding of the antibody to the one or more SARS CoV-2 antigens is indicative of the presence of SARS CoV-2 in the sample.

In some embodiments, the antibody or antigen-binding fragment thereof is conjugated to a label. In some embodiments, the step of detecting comprises contacting a secondary antibody with the antibody or antigen-binding fragment thereof and wherein the secondary antibody comprises a label. In some embodiments, the label includes a fluorescent label, a chemiluminescent label, a radiolabel, and an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and an affinity purification column.

In yet another aspect, this disclosure additionally provides a pharmaceutical composition comprising a first isolated anti-SARS-CoV-2 antibody or antigen-binding fragment thereof described herein, and a second isolated anti-SARS-CoV-2 antibody or antigen-binding fragment thereof that binds specifically to the SARS-CoV-2 antigen.

In some embodiments, the first anti-SARS-CoV-2 antibody or antigen-binding fragment thereof and the second anti-SARS-CoV-2 antibody or antigen-binding fragment thereof bind to different epitopes of the SARS-CoV-2 antigen. In some embodiments, a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

In some embodiments, the second anti-SARS-CoV-2 antibody comprises six CDRs (HCDR1, HCDR2, HCDR3, LCDR1, LCDR2, and LCDR3) of Sotrovimab having respective amino acid sequences of SEQ ID NOs: 37-42.

In some embodiments, the second anti-SARS-CoV-2 antibody comprises HCVR of Sotrovimab having the amino acid sequence of SEQ ID NO: 43 and/or LCVR of Sotrovimab having the amino acid sequence of SEQ ID NO: 44.

In some embodiments, the second anti-SARS-CoV-2 antibody comprises or is Sotrovimab.

In another aspect, this disclosure further provides a method of neutralizing SARS-CoV-2 or treating a SARS-CoV-2 infection in a subject. In some embodiments, the method comprises administering to a subject in need thereof a therapeutically effective amount of a first isolated anti-SARS-CoV-2 antibody or antigen-binding fragment thereof described herein, and a therapeutically effective amount of a second isolated anti-SARS-CoV-2 antibody or antigen-binding fragment thereof that binds specifically to the SARS-CoV-2 antigen.

In some embodiments, the first anti-SARS-CoV-2 antibody or antigen-binding fragment thereof and the second anti-SARS-CoV-2 antibody or antigen-binding fragment thereof bind to different epitopes of the SARS-CoV-2 antigen. In some embodiments, a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

In some embodiments, the second anti-SARS-CoV-2 antibody comprises six CDRs (HCDR1, HCDR2, HCDR3, LCDR1, LCDR2, and LCDR3) of Sotrovimab.

In some embodiments, the second anti-SARS-CoV-2 antibody comprises HCVR and/or LCVR of Sotrovimab.

In some embodiments, the second anti-SARS-CoV-2 antibody comprises or is Sotrovimab.

In another aspect, this disclosure provides an isolated polypeptide comprising an amino acid sequence having at least 75% sequence identity to the amino acid sequence of SEQ ID NO: 13 or having the amino acid sequence of SEQ ID NO: 13.

Also within the scope of this disclosure are a nucleic acid molecule encoding the polypeptide, a vector comprising the nucleic acid molecule, and a host cell comprising the nucleic acid or the vector, as described herein.

Further provided in this disclosure is a virus-like particle comprising the polypeptide, the nucleic acid, or the vector, as described herein.

In another aspect, this disclosure provides an immunogenic composition for stimulating an immune response in a subject in need thereof. In some embodiments, the immunogenic composition comprises the polypeptide, the nucleic acid, the vector, the virus-like particle, as described herein, or a combination thereof.

In some embodiments, the immunogenic composition is capable of inducing production of at least two anti-SARS-CoV2 antibodies that bind specifically to different epitopes on a spike protein of a SARS-CoV2 variant.

In another aspect, this disclosure provides a method of stimulating an immune response in a subject in need thereof. In some embodiments, the method comprises administrating to the subject an effective amount of the polypeptide, the nucleic acid, the vector, the virus-like particle, the immunogenic composition, as described herein, or a combination thereof.

In another aspect, this disclosure provides a method of neutralizing SARS-CoV-2 in a subject in need thereof. In some embodiments, the method comprises administrating to the subject an effective amount of the polypeptide, the nucleic acid, the vector, the virus-like particle, the immunogenic composition, as described herein, or a combination thereof.

In another aspect, this disclosure provides a method of treating a SARS-CoV-2 infection in a subject in need thereof. In some embodiments, the method comprises administrating to the subject an effective amount of the polypeptide, the nucleic acid, the vector, the virus-like particle, the immunogenic composition, as described herein, or a combination thereof.

In some embodiments, the immunogenic composition is administered to the subject in one or more doses or in two or more times.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows receptor binding domains (RBDs) of Omicron subvariants that are highly mutated, as compared to CoV2-WT-Wuhan. The amino acid sequence of the spike from 229 to 436 representing the RBD of CoV2 Omicron sub-variants was compared with ancestral CoV2Wuhan-1 to show the level of mutation acquired in continuously evolving Omicron variants.

The identity with the ancestral amino acid sequence and the mutated amino acids compared to the CoV2-Wuhan-1 variant are indicated. The sequences as shown (Wuhan1, BA.3, BA.1, BA.1.1, BA.2, BA.2.12.1, BA4/5, BA.4.6, BQ.1.1, BQ.1, BA.2.75, and XBB) have SEQ ID NOs: 14-25, respectively. The region between amino acids 446 and 506 plays a crucial role in ACE2 receptor binding to target cells and is known as the receptor-binding motif (RBM).

FIG. 2 shows a graphical representation of IC50 neutralization endpoints of nine plasma from subjects who received COVID-19 vaccines and who were also infected with CoV2 against pseudoviruses expressing Spikes from six different CoV2 variants (CoV2-D614G, Alpha, Beta, Gamma, Delta, and BA.1 Omicron).

FIG. 3 tabulates IC50 neutralization endpoints of a plasma of a COVID-19 vaccinated-infected patient before and after absorption of antibodies directed against conserved RBD-neutralizing epitopes. The plasma was passed over a column containing immobilized gp70-RBDwt to remove all reactive antibodies. The upper panels represent binding of original and absorbed plasma to RBD_{wt} and to an N-terminal domain fragment of the Spike protein (NTD), and demonstrates complete removal of all of the RBD-reactive antibodies and retention of the great majority of the NTD-reactive antibodies. The lower panel shows the IC50 of the unabsorbed and absorbed plasma against CoV2-D614G, delta, and omicron BA.1 variants, and demonstrates that the great majority of the neutralizing activity of this plasma against all of these variants was lost upon absorption of the RBDwt-reactive antibodies. This shows that the potent neutralizing activity of this plasma was predominantly directed against RBD epitopes that were conserved between the Wuhan1 and Omicron sequences.

FIG. 4 shows RBD-WT and RBD-BA.1-bound memory B-cell sorting strategies. Ten million PBMCs were treated with Fc-block, mouse anti-human CD3-BUV395, CD4-BUV395, CD8-BUV395, CD14BUV-395, IgDBUV-395, IgG-FITC, CD19-PE-Cy7 antibodies, RBD-CoV2-WT-AF647, RBD-BA.1-AF594, and DAPI and analyzed using BD FACS Aria II to sort the RBD-AF647+AF594+ double positive single memory B cells by sequential gating. Single-cell populations were generated by plotting the cell populations of SSC-H/FSC-A and FSC-H/SSC-A cells. DAPI-negative single-cell populations were gated to live cells. DAPI negative BUV395 positive cells were excluded to remove non-B and naïve B cells. BUV395 negative DAPI negative cells were gated for FITC- and PE-positive cells to gate IgG-expressing memory B cells, which were further selected for AF-647 and AF-594 positive cells to sort the RBD-CoV2-WT+ RBD-Omicron-WT+ double-positive single memory B cells.

FIG. 5 shows neutralization potency of a sorted memory B cell culture supernatant. Memory B cell culture supernatants from 28 different wells positive for binding with RBD-WT and RBD-BA.1 were tested for neutralization against CoV2-WT and BA.1 pseudoviruses. Only one well, P1A7, potently neutralized both CoV2-WT and BA.1.

FIGS. 6A and 6B show IGVH and IGVK analysis of the ACOVIMAB monoclonal antibody. FIG. 6A shows IGVH analysis of the ACOVIMAB monoclonal antibody. IGVH derived from P1A7 well was cloned into the IGG1 heavy chain expression vector and analyzed using the IMGT database for VDJ gene usage, framework (FR) and complementarity determining region (CDR), V-D and DJ-junction, CDR3 length, and percentage mutations in FR1, CDR1, FR2, CDR2, FR3, and CDR3, separately and as a whole. The level of amino acid mutation in Acovimab IGVH compared to that in the VH1-58 germline sequence was calculated manually, as shown by the alignment of the two sequences performed by ClustaW. FIG. 6B shows IGVK analysis of the ACOVIMAB monoclonal antibody. IGVK derived from P1A7 well was cloned into the kappa light chain expression vector and analyzed using the IMGT database for VJ gene usage, framework (FR) and complementarity determining region (CDR), VJ-junction, CDR3 length, and percentage mutations in FR1, CDR1, FR2, CDR2, FR3, and CDR3, separately and as a whole. The level of amino acid mutation in Acovimab IGVK compared to that in the VK3-20 germline sequence was calculated manually, as shown by the alignment of the two sequences performed by ClustaW. SEQ ID NOs assigned to the sequences in FIGS. 6A and 6B are provided in Table 1.

FIG. 7 shows a comparative binding analysis of ACOVIMAB and other RBD mAbs to CoV2-WT and BA.1 spike. Anti-RBD mAbs binding with CoV2-B.1-WT and BA.1 spike was performed by ELISA. Binding curves with titration of mAbs were generated to calculate the area under the curve (AUC) to compare mAb binding to CoV2-B.1-WT and BA.1 spike.

FIG. 8 shows that anti-RBD mAbs competition patterns were determined by ELISA, using a checkerboard in which the RBD antigen was plated out and probed for reactivity with each of the biotinylated mAbs in the absence and presence of a large excess (100 μg/ml) of each competing mAb. Two major competition groups were identified, together with several minor groups with overlapping properties.

FIGS. 9A and 9B show anti-RBD mAbs footprints on RBD. Binding epitopes for all eight anti-RBD therapeutic mAbs were determined from their cryo-EM structures. The Acovimab binding region is predicted to be similar to that of Tixagevimab, based on their sequence derivation from the same germline sequences and their efficient competition for RBD binding. The sequences in FIG. 9A for B.1 and BQ.1.1 are assigned with SEQ ID NO: 26 and 27, respectively. The sequences in FIG. 9B for IGHV1-58 (SEQ ID NO: 28) and IGKV3-20 (SEQ ID NO: 29), Tixagevimab (HCVR: SEQ ID NO: 30 and LCVR: SEQ ID NO: 31), and Acovimab (HCVR: SEQ ID NO: 32 and LCVR: SEQ ID NO: 33), respectively.

FIG. 10 shows anti-RBD mAbs ACE2-RBD-B.1 binding. 8 out of 9 anti-RBD mAbs competed completely with soluble Hu-ACE2-biotinylated for binding with RBD-B.1 coated on the ELISA plate. All mAbs except Sotrovimab compete strongly with ACE2 binding, consistent with their binding to the receptor-binding domain (RBM). Sotrovimab gave <50% competition at the highest concentration tested, consistent with binding to a site outside of the RBM and indicating an alternative mechanism for neutralization by this mAb.

FIGS. 11A and 11B show Combination Index (CI) and synergistic neutralization of Acovimab and Sotrovimab. FIG. 11A shows Combination Index (CI) calculated for the Acovi+Sotrovi mAb combination (CI value <1 shows a synergistic effect of the two mAbs). FIG. 11B shows synergistic neutralization of Acovimab and Sotrovimab for BQ.1.1.

FIG. 12 shows an example chimeric RBD Immunogen design for effective induction of Sotrovimab and Acovimab-like antibodies. The sequences as shown for CoV1 RBD, Wuhan Hu, XBB.1.5, and the chimeric immunogen are assigned with SEQ ID NOs: 34, 35, 36, and 13, respectively.

### DETAILED DESCRIPTION

SARS-CoV-2 represents a serious public health concern. Methods to diagnose and treat persons who are infected with SARS-CoV-2 provide the opportunity to either prevent or control further spread of infection by SARS-CoV-2. This disclosure provides novel isolated and recombinantly modified anti-SARS-CoV-2 neutralizing antibodies or antigen-binding fragments thereof that bind specifically to an antigen of SARS-CoV-2 Omicron and other variants. Also provided in this disclosure are novel combinations of anti-SARS-CoV-2 antibodies that exhibit unexpected synergistic effects in neutralizing SARS-CoV-2 variants. Furthermore, this disclosure provides novel chimeric immunogens for inducing effective immune responses against SARS-CoV-2 infections.

### Neutralizing Anti-SARS-CoV-2 Antibodies

Antibodies

In one aspect, this disclosure provides novel isolated and recombinantly modified anti-SARS-CoV-2 neutralizing antibodies or antigen-binding fragments thereof. These antibodies refer to a class of broadly neutralizing antibodies that neutralize SARS-CoV-2 Omicron and other variants.

In one aspect, this disclosure provides an isolated anti-SARS-CoV-2 antibody or antigen-binding fragment thereof that binds specifically to a SARS-CoV-2 antigen. In some embodiments, the SARS-CoV-2 antigen comprises a portion of a spike (S) polypeptide, such as a S polypeptide of a human or an animal SARS-CoV-2. In some embodiments, the SARS-CoV-2 antigen comprises the receptor-binding domain (RBD) of the S polypeptide. In some embodiments, the RBD comprises amino acids 319-541 of the S polypeptide.

The spike protein (S) is important because it is present on the outside of intact SARS-CoV-2, and mediates binding to the ACE2 receptor and fusion of the virus with the membrane of target cells, steps essential for infection. Thus, it presents a target for reagents that can be used to inhibit the virus from infecting a cell. A representative S protein amino acid sequence is set forth in Accession ID: NC 045512.2. The total length of SARS-CoV-2 S is 1273 amino acids and consists of a signal peptide (amino acids 1-13) located at the N-terminus, the S1 subunit (14-685 residues), and the S2 subunit (686-1273 residues); the last two regions are responsible for receptor binding and membrane fusion, respectively. In the S1 subunit, there is an N-terminal domain (14-305 residues) and a receptor-binding domain (RBD, 319-541 residues). The S2 subunit comprises the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues). S protein trimers visually form a characteristic bulbous, crown-like halo surrounding the viral particle. Based on the structure of coronavirus S protein monomers, the S1 and S2 subunits form the bulbous head and stalk region.

In some embodiments, the antibody or antigen-binding fragment thereof comprises: three heavy chain complementarity determining regions (HCDRs) (HCDR1, HCDR2, and HCDR3) of a heavy chain variable region (HCVR) having the amino acid sequence of SEQ ID NO: 7; and three light chain CDRs (LCDR1, LCDR2, and LCDR3) of a light chain variable region (LCVR) having the amino acid sequence of SEQ ID NO: 8.

In some embodiments, HCDR1, HCDR2, HCDR3, LCDR1, LCDR2, and LCDR3 comprise respective amino acid sequences of SEQ ID NOs: 1-6.

In some embodiments, the antibody or antigen-binding fragment thereof comprises a HCVR having the amino acid sequence of SEQ ID NO: 7 or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 7.

In some embodiments, the antibody or antigen-binding fragment thereof comprises a LCVR having the amino acid sequence of SEQ ID NO: 8 or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 8.

In some embodiments, the antibody or antigen-binding fragment thereof comprises a HCVR having the amino acid sequence of SEQ ID NO: 7, and a LCVR having the amino acid sequence of SEQ ID NO: 8.

The antibody or antigen-binding fragment thereof comprises a heavy chain having the amino acid sequence of SEQ ID NO: 9 or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 9.

In some embodiments, the antibody or antigen-binding fragment thereof comprises a light chain having the amino acid sequence of SEQ ID NO: 10 or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 10.

In some embodiments, the antibody or antigen-binding fragment thereof comprises a heavy chain having the amino acid sequence of SEQ ID NO: 9, and a light chain having the amino acid sequence of SEQ ID NO: 10.

In some embodiments, the antibody or antigen-binding fragment thereof comprises HCDR1, HCDR2, HCDR3, LCDR1, LCDR2, and LCDR3 of ACOVIMAB.

In some embodiments, the antibody or antigen-binding fragment thereof comprises a HCVR having the amino acid sequence of the HCVR of ACOVIMAB or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of the HCVR of ACOVIMAB.

In some embodiments, the antibody or antigen-binding fragment thereof comprises a LCVR having the amino acid sequence of the LCVR of ACOVIMAB or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of the LCVR of ACOVIMAB.

In some embodiments, the antibody or antigen-binding fragment thereof comprises a heavy chain having the amino acid sequence of the heavy chain of ACOVIMAB or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of the heavy chain of ACOVIMAB.

In some embodiments, the antibody or antigen-binding fragment thereof comprises a light chain having the amino acid sequence of the light chain of ACOVIMAB or having an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of the light chain of ACOVIMAB.

In some embodiments, the antibody or antigen-binding fragment thereof is ACOVIMAB.

TABLE 1
Representative sequences of the anti-SARS-CoV-2 antibodies

| SEQ | | |
|---|---|---|
| ID | | |
| NO | SEQUENCE | INFORMATION |
| 1 | GFTFTGT | HCDR1 |
| 2 | IVVGSGET | HCDR2 |
| 3 | AAPYCSGGTCMDGFDL | HCDR3 |
| 4 | QSVRSNY | LCDR1 |
| 5 | AAS | LCDR2 |
| 6 | QQYDRSPWT | LCDR3 |
| 7 | EVQLVESGPEVKKPGTSVKVSCKASGFTFTTTSSVQWVRQARG | ACOVIMAB_ |
| | QRLEWIGWIVVGSGETEYAQNFQDRVTITSDMSTTTAYMEMSS | IGVH |
| | LRSEDSAFYYCAAPYCSGGTCMDGFDLWGQGTLVSVSS | p9952 |
| 8 | EIVLTQSPGTLSLSPGERATLSCRASQSVRSNYLGWYQQKPGQA | ACOVIMAB_ |
| | PRLLIYAASSRATGIPDRFSGSGSGTDFTLTISRLEPEDFAVYYCQ | IGVK |
| | QYDRSPWTFGQGTKVESK | p9953 |
| 9 | ASTKGPSVFPLAPSSKSTSGGTAALGCLVKDYFPEPVTVSWNSG | HC |
| | ALTSGVHTFPAVLQSSGLYSLSSVVTVPSSSLGTQTYICNVNHK | |
| | PSNTKVDKRVEPKSCDKTHTCPPCPAPELLGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| 10 | RTVAAPSVFIFPPSDEQLKSGTASVVCLLNNFYPREAKVQWKV | LC |
| | DNALQSGNSQESVTEQDSKDSTYSLSSTLTLSKADYEKHKVYA | |
| | CEVTHQGLSSPVTKSFNRGEC | |
| 11 | GAAGTGCAGCTCGTGGAGTCTGGGCCTGAGGTGAAGAAGCC | ACOVIMAB_ |
| | TGGGACCTCAGTGAAGGTCTCCTGCAAGGCCTCTGGATTCAC | IGVH |
| | CTTTACTACGACGTCTTCTGTGCAGTGGGTGCGGCAGGCTCG | |
| | TGGACAACGCCTTGAGTGGATAGGCTGGATCGTCGTTGGCA | |
| | GTGGTGAGACAGAGTACGCACAGAACTTCCAAGACAGAGTC | |
| | ACCATTACCAGCGACATGTCCACAACCACAGCCTACATGGA | |
| | GATGAGCAGCCTGAGATCCGAGGACTCGGCCTTTTATTATTG | |
| | TGCGGCTCCCTATTGTAGTGGTGGAACCTGCATGGACGGTTT | |
| | TGATCTCTGGGGCCAAGGGACATTGGTCAGCGTCTCCTCAG | |
| 12 | GAAATTGTGTTGACGCAGTCTCCAGGCACCCTGTCTTTGTCT | ACOVIMAB_ |
| | CCAGGGGAAAGAGCCACCCTCTCCTGCAGGGCCAGTCAGAG | IGVK |
| | TGTTAGAAGTAACTACTTAGGCTGGTACCAGCAGAAACCTG | |
| | GCCAGGCTCCCAGGCTCCTCATCTATGCTGCATCCAGCAGGG | |
| | CCACTGGCATCCCAGACAGGTTCAGTGGCAGTGGATCTGGG | |
| | ACAGACTTCACTCTCACCATCAGTAGACTGGAGCCTGAAGAT | |
| | TTTGCAGTGTATTACTGTCAGCAGTATGATAGGTCACCGTGG | |
| | ACGTTCGGCCAAGGGACCAAGGTGGAAAGCAAAC | |
| 13 | FPNITNLCPFGEVFNATKFPSVYAWERKKISNCVADYSVLYNST | Chimeric |
| | FFSTFKCYGVSATKLNDLCFSNVYADSFVVKGDDVRQIAPGQT | immunogen |
| | GVIADYNYKLPDDFMGCVLAWNTRNIDATSTGNYNYLYRLFR | |
| | KSKLKPFERDISTEIYQAGNKPCNGVAGPNCYSPLQSYGFRPTY | |
| | GVGHQPYRVVVLSFELLHAPATVCGPK | |

A person of ordinary skill in the art will understand that various CDR numbering schemes (such as the Kabat, Chothia, Enhanced Chothia, IMGT, AHoAbM, Contact numbering schemes) can be used herein to determine CDR positions.

In some embodiments, the antibody or antigen-binding fragment thereof comprises a first target binding site that specifically binds to an epitope within the S polypeptide, and a second target binding site that binds to a different epitope on the S polypeptide or on a different molecule. In some embodiments, the multivalent antibody is a bivalent or bispecific antibody.

In some embodiments, the antibody or the antigen-binding fragment thereof further comprises a variant Fc constant region. In some embodiments, the antibody possesses an Fc LS mutation (M428L/N434S) that confers enhanced binding to the neonatal Fc receptor and results in an extended half-life.

In some embodiments, the antibody is a monoclonal antibody. In some embodiments, the antibody is a chimeric antibody, a humanized antibody, or a humanized monoclonal antibody. In some embodiments, the antibody is a single-chain antibody, Fab or Fab2 fragment.

In some embodiments, the antibody or the antigen-binding fragment thereof further comprises a variant Fc constant region. The antibody can be a monoclonal antibody. In some embodiments, the antibody can be a chimeric antibody, a humanized antibody, or a humanized monoclonal antibody. In some embodiments, the antibody can be a single-chain antibody, Fab or Fab2 fragment.

In some embodiments, the antibody or antigen-binding fragment thereof can be detectably labeled or conjugated to a toxin, a therapeutic agent, a polymer (e.g., polyethylene glycol (PEG)), a receptor, an enzyme, or a receptor ligand. For example, an antibody of the present disclosure may be coupled to a toxin (e.g., a tetanus toxin). Such antibodies may be used to treat animals, including humans, that are infected with the virus that is etiologically linked to SARS-CoV-2. The toxin-coupled antibody is thought to bind to a portion of a spike protein presented on an infected cell, and then kill the infected cell.

In another example, an antibody of the present disclosure may be coupled to a detectable tag. Such antibodies may be used within diagnostic assays to determine if an animal, such as a human, is infected with SARS-CoV-2. Examples of detectable tags include fluorescent proteins (i.e., green fluorescent protein, red fluorescent protein, yellow fluorescent protein), fluorescent markers (i.e., fluorescein isothiocyanate, rhodamine, texas red), radiolabels (i.e., 3H, 32P, 1251), enzymes (i.e., 0-galactosidase, horseradish peroxidase, β-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs or portions thereof, are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells and using PCR primers containing random sequences to encode the highly variable CDR3 regions and to accomplish rearrangement in vitro, as described by Hoogenboom and Winter, J. Mol. Biol., 227: 381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this invention are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the)(BLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used. (See www.ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions, and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17: 176-180).

Fc Region Variants

The variable regions of the antibody described herein can be linked (e.g., covalently linked or fused) to an Fc, e.g., an IgGl, IgG2, IgG3 or IgG4 Fc, which may be of any allotype or isoallotype, e.g., for IgGl: Glm, Glml(a), Glm2(x), Glm3(f), Glm17(z); for IgG2: G2m, G2m23(n); for IgG3: G3m, G3m21(g1), G3m28(g5), G3ml l(b0), G3m5(b1), G3ml3(b3), G3ml4(b4), G3ml0(b5), G3ml5(s), G3ml6(t), G3m6(c3), G3m24(c5), G3m26(u), G3m27(v); and for K: Km, Kml, Km2, Km3 (see, e.g., Jefferies et al. (2009) mAbs 1: 1). In some embodiments, the antibodies variable regions described herein are linked to an Fc that binds to one or more activating Fc receptors (FcγI, Fcγlla, or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant, or derivative of the constant region. Suitable immunoglobulins include IgGl, IgG2, IgG3, IgG4, and other classes such as IgA, IgD, IgE, and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. In some embodiments, an antibody of this invention has an Fc region other than that of a wild type IgA1. The antibody can have an Fc region from that of IgG (e.g., IgGl, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgGl, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgGl. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgGl. Specific examples of variant Fc domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered Clq binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Other Fc modifications that can be made to Fcs are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRllb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art, including but not limited to equilibrium methods (e.g., ELISA, radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al, 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgGl/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgGl/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgG1 for FcγRl, FcγRII, FcγRIII, and FcRn have been mapped, and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L, and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include S298A/E333A/L334A, S239D/I332E, S239D/I332E/A330L, L235V/F243L/R292P/Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A.

In some embodiments, an Fc is chosen that has reduced complement fixation. An exemplary Fc, e.g., IgGl Fc, with reduced complement fixation, has the following two amino acid substitutions: A330S and P331S.

In some embodiments, an Fc is chosen that has essentially no effector function, i.e., it has reduced binding to FcγRs and reduced complement fixation. An exemplary Fc, e.g., IgGl Fc, that is effectorless, comprises the following five mutations: L234A, L235E, G237A, A330S, and P331S.

When using an IgG4 constant domain, it is usually preferable to include the substitution S228P, which mimics the hinge sequence in IgG1 and thereby stabilizes IgG4 molecules.

Multivalent Antibodies

In one embodiment, the antibodies of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind to the same or different molecules (e.g., may bind to different ligands or different antigens, or different epitopes or positions on the same antigen). See, for example, U.S.P.N. 2009/0130105. In each case, at least one of the binding sites will comprise an epitope, motif or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities, such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “hetero-conjugate” antibodies. For example, one of the antibodies in the hetero-conjugate can be coupled to avidin, and the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

The present disclosure also encompasses a human monoclonal antibody described herein conjugated to a therapeutic agent, a polymer, a detectable label, or an enzyme. In one embodiment, the therapeutic agent is a cytotoxic agent. In one embodiment, the polymer is PEG.

Nucleic Acids, Expression Cassettes, and Vectors

The present disclosure provides isolated nucleic acid segments that encode the polypeptides, peptide fragments, and coupled proteins of the invention. The nucleic acid segments of the invention also include segments that encode for the same amino acids due to the degeneracy of the genetic code. For example, the amino acid threonine is encoded by ACU, ACC, ACA, and ACG and is therefore degenerate. It is intended that the invention includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of the invention include both naturally occurring sequences as well as mutant forms.

The nucleic acid segments of the invention may be contained within a vector. A vector may include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

The nucleic acid segment in the vector can be under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the invention may also be inserted into an expression vector. Typically, an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce a nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA-modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

The invention also provides an expression cassette that contains a nucleic acid sequence capable of directing expression of a particular nucleic acid segment of the invention, either in vitro or in a host cell. Also, a nucleic acid segment of the invention may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment, and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The invention also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the invention into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

Methods of Production

In another aspect, this disclosure also provides (i) a nucleic acid molecule encoding a polypeptide chain of the antibody or antigen-binding fragment thereof described herein; (ii) a vector comprising the nucleic acid molecule as described; and (iii) a host cell comprising the vector as described. Also provided is a method for producing a polypeptide, comprising: culturing the host cell in a medium under conditions permitting expression of a polypeptide encoded by the vector and assembling of an antibody or fragment thereof; and isolating the antibody or fragment from the cultured cell or the medium of the cell.

Antibodies may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Compositions and Kits

The antibodies of this disclosure represent an excellent way for the development of antiviral therapies either alone or in antibody cocktails with additional anti-SARS-CoV-2 virus antibodies for the treatment of human SARS-CoV-2 infections in humans.

In another aspect, the present disclosure provides a pharmaceutical composition comprising the antibodies described herein formulated together with a pharmaceutically acceptable carrier. The composition may optionally contain one or more additional pharmaceutically active ingredients, such as another antibody or a therapeutic agent.

In some embodiments, the pharmaceutical comprises two or more of the antibody or antigen-binding fragment thereof described herein, such as any combinations of the antibody or antigen-binding fragment thereof comprising a heavy chain and a light chain that comprise the respective amino acid sequences described herein.

The pharmaceutical compositions of the invention also can be administered in a combination therapy with, for example, another immune-stimulatory agent, an antiviral agent, a vaccine, etc. In some embodiments, a composition comprises an antibody of this invention at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine, or interferon. In some embodiments, the interferon is an interferon-α or an interferon-β.

Also within the scope of this disclosure is use of the pharmaceutical composition in the preparation of a medicament for the diagnosis, prophylaxis, treatment, or combination thereof of a condition resulting from a SARS-CoV-2.

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

In some embodiments, a pharmaceutical composition is suitable for intravenous, intramuscular, subcutaneous, parenteral, spinal, or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the present disclosure can be administered via a non-parenteral route, such as a topical, epidermal, or mucosal route of administration, e.g., intranasally, orally, vaginally, rectally, sublingually, or topically.

The pharmaceutical compositions of the invention may be prepared in many forms that include tablets, hard or soft gelatin capsules, aqueous solutions, suspensions, liposomes, and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers, or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer, a pressurized pack, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in a unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated, and the age and condition of the patient. Ultimately the attendant health care provider may determine a proper dosage. In addition, a pharmaceutical composition may be formulated as a single unit dosage form.

The pharmaceutical composition of the present disclosure can be in the form of sterile aqueous solutions or dispersions. It can also be formulated in a microemulsion, liposome, or other ordered structure suitable to high drug concentration.

An antibody of the present disclosure can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens can be adjusted to provide the optimum desired response (e.g., a therapeutic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration is required. For administration of the antibody, the dosage ranges from about 0.0001 to 800 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml, and in some methods, about 25-300 μg/ml. A “therapeutically effective dosage” of an antibody of the invention preferably results in a decrease in severity of disease symptoms, an increase in frequency and duration of disease symptom-free periods, or a prevention of impairment or disability due to the disease affliction. For example, for the treatment of SARS-CoV-2 infection in a subject, a “therapeutically effective dosage” preferably inhibits SARS-CoV-2 virus replication or uptake by host cells by at least about 20%, more preferably by at least about 40%, even more preferably by at least about 60%, and still more preferably by at least about 80% relative to untreated subjects. A therapeutically effective amount of a therapeutic compound can neutralize SARS-CoV-2 virus, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene-vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparati (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the human monoclonal antibodies of the invention described herein can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs. See, e.g., U.S. Pat. Nos. 4,522,811; 5,374,548; 5,416,016; and 5,399,331; V. V. Ranade (1989) Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273.

In some embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor-mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can also be delivered in a vesicle, in particular, a liposome (see, for example, Langer (1990) Science 249: 1527-1533).

The use of nanoparticles to deliver the antibodies of the present disclosure is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal, intramuscular injections, drip infusions, etc. These injectable preparations may be prepared by methods publicly known. For example, the injectable preparations may be prepared, e.g., by dissolving, suspending, or emulsifying the antibody or its salt described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pens and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but certainly are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.) and the HUMIRA™ Pen (Abbott Labs, Abbott Park, IL), to name only a few.

Advantageously, the pharmaceutical compositions for oral or parenteral use described herein are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

In another aspect, this disclosure provides a kit comprising a pharmaceutically acceptable dose unit of the antibody or antigen-binding fragment thereof or the pharmaceutical composition as described herein. Also within the scope of this disclosure is a kit for the diagnosis, prognosis, or monitoring of treatment of SARS-CoV-2 in a subject, comprising: the antibody or antigen-binding fragment thereof as described; and a least one detection reagent that binds specifically to the antibody or antigen-binding fragment thereof.

In some embodiments, the kit also includes a container that contains the composition and optionally informational material. The informational material can be descriptive, instructional, marketing, or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### Methods of Use of the Antibodies

Methods of Treatment

The antibodies, compositions, and formulations described herein can be used to neutralize SARS-CoV-2 virus, thereby treating or preventing SARS-CoV-2 infections.

Accordingly, in one aspect, this disclosure further provides a method of neutralizing SARS-CoV-2 in a subject. In some embodiments, the method may include administering to a subject in need thereof a therapeutically effective amount of the antibody or antigen-binding fragment thereof or a therapeutically effective amount of the pharmaceutical composition, as described herein.

In another aspect, this disclosure additionally provides a method of preventing or treating a SARS-CoV-2 infection. In some embodiments, the method may include administering to a subject in need thereof a therapeutically effective amount of the antibody or antigen-binding fragment thereof or a therapeutically effective amount of the pharmaceutical composition, as described herein.

The neutralizing of the SARS-CoV-2 virus can be done via (i) inhibiting SARS-CoV-2 virus binding to a target cell; (ii) inhibiting SARS-CoV-2 virus uptake by a target cell; (iii) inhibiting SARS-CoV-2 virus replication; and (iv) inhibiting SARS-CoV-2 virus particles release from infected cells. One skilled in the art possesses the ability to perform any assay to assess neutralization of SARS-CoV-2 virus.

Notably, the neutralizing properties of antibodies may be assessed by a variety of tests, which all may assess the consequences of (i) inhibition of SARS-CoV-2 virus binding to a target cell; (ii) inhibition of SARS-CoV-2 virus uptake by a target cell; (iii) inhibition of SARS-CoV-2 virus replication; and (iv) inhibition of SARS-CoV-2 virus particles release from infected cells. In other words, implementing different tests may lead to the observation of the same consequence, i.e., the loss of infectivity of the SARS-CoV-2 virus. Thus, in one embodiment, the present disclosure provides a method of neutralizing a SARS-CoV-2 virus in a subject comprising administering to the subject a therapeutically effective amount of the antibody of the present disclosure.

Another aspect of the present disclosure provides a method of treating a SARS-CoV-2-related disease. Such a method includes therapeutic (following SARS-CoV-2 infection) and prophylactic (prior to SARS-CoV-2 exposure, infection, or pathology). For example, therapeutic and prophylactic methods of treating an individual for a SARS-CoV-2 infection include treatment of an individual having or at risk of having a SARS-CoV-2 infection or pathology, treating an individual with a SARS-CoV-2 infection, and methods of protecting an individual from a SARS-CoV-2 infection, to decrease or reduce the probability of a SARS-CoV-2 infection in an individual, to decrease or reduce susceptibility of an individual to a SARS-CoV-2 infection, or to inhibit or prevent a SARS-CoV-2 infection in an individual, and to decrease, reduce, inhibit or suppress transmission of a SARS-CoV-2 from an infected individual to an uninfected individual. Such methods include administering an antibody or a composition comprising the antibody disclosed herein to therapeutically or prophylactically treat (vaccinate or immunize) an individual having or at risk of having a SARS-CoV-2 infection or pathology. Accordingly, methods can treat the SARS-CoV-2 infection or pathology, or provide the individual with protection from infection (e.g., prophylactic protection).

In one embodiment, a method of treating a SARS-CoV-2-related disease comprises administering to an individual in need thereof an antibody or therapeutic composition disclosed herein in an amount sufficient to reduce one or more physiological conditions or symptoms associated with a SARS-CoV-2 infection or pathology, thereby treating the SARS-CoV-2-related disease.

In one embodiment, an antibody or therapeutic composition disclosed herein is used to treat a SARS-CoV-2-related disease. Use of an antibody or therapeutic composition disclosed herein treats a SARS-CoV-2-related disease by reducing one or more physiological conditions or symptoms associated with a SARS-CoV-2 infection or pathology. In aspects of this embodiment, administration of an antibody or therapeutic composition disclosed herein is in an amount sufficient to reduce one or more physiological conditions or symptoms associated with a SARS-CoV-2 infection or pathology, thereby treating the SARS-CoV-2-based disease. In other aspects of this embodiment, administration of an antibody or therapeutic composition disclosed herein is in an amount sufficient to increase, induce, enhance, augment, promote or stimulate SARS-CoV-2 clearance or removal; or decrease, reduce, inhibit, suppress, prevent, control, or limit transmission of SARS-CoV-2 to another individual.

One or more physiological conditions or symptoms associated with a SARS-CoV-2 infection or pathology will respond to a method of treatment disclosed herein. The symptoms of SARS-CoV-2 infection or pathology vary, depending on the phase of infection.

In some embodiments, the method may include administering to the subject a second therapeutic agent. In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine, or an interferon. In some embodiments, the interferon is an interferon-α or an interferon-β.

In some embodiments, the antibody or antigen-binding fragment thereof is administered before, after, or concurrently with the second therapeutic agent or therapy. In some embodiments, the antibody or antigen-binding fragment thereof is administered to the subject intravenously, subcutaneously, or intraperitoneally. In some embodiments, the antibody or antigen-binding fragment thereof is administered prophylactically or therapeutically.

The antibodies described herein can be used together with one or more of other anti-SARS-CoV-2 virus antibodies to neutralize SARS-CoV-2 virus, thereby preventing or treating SARS-CoV-2 infections.

Administration Regimens

According to certain embodiments, a single dose of an anti-SARS-CoV-2 antibody of the invention (or a pharmaceutical composition comprising a combination of an anti-SARS-CoV-2 antibody and any of the additional therapeutically active agents mentioned herein) may be administered to a subject in need thereof. According to certain embodiments of the present disclosure, multiple doses of an anti-SARS-CoV-2 antibody (or a pharmaceutical composition comprising a combination of an anti-SARS-CoV-2 antibody and any of the additional therapeutically active agents mentioned herein) may be administered to a subject over a defined time course. The methods according to this aspect of the invention comprise sequentially administering to a subject multiple doses of an anti-SARS-CoV-2 antibody of the invention. As used herein, “sequentially administering” means that each dose of anti-SARS-CoV-2 antibody is administered to the subject at a different point in time, e.g., on different days separated by a predetermined interval (e.g., hours, days, weeks, or months). The present disclosure includes methods that comprise sequentially administering to the patient a single initial dose of an anti-SARS-CoV-2 antibody, followed by one or more secondary doses of the anti-SARS-CoV-2 antibody, and optionally followed by one or more tertiary doses of the anti-SARS-CoV-2 antibody.

The terms “initial dose,” “secondary doses,” and “tertiary doses,” refer to the temporal sequence of administration of the anti-SARS-CoV-2 antibody of the invention. Thus, the “initial dose” is the dose, which is administered at the beginning of the treatment regimen (also referred to as the “baseline dose”); the “secondary doses” are the doses, which are administered after the initial dose; and the “tertiary doses” are the doses which are administered after the secondary doses. The initial, secondary, and tertiary doses may all contain the same amount of anti-SARS-CoV-2 antibody, but generally may differ from one another in terms of frequency of administration. In some embodiments, however, the amount of anti-SARS-CoV-2 antibody contained in the initial, secondary and/or tertiary doses varies from one another (e.g., adjusted up or down as appropriate) during the course of treatment. In some embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments, each secondary and/or tertiary dose is administered 1 to 48 hours (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) after the immediately preceding dose. The phrase “the immediately preceding dose,” as used herein, means, in a sequence of multiple administrations, the dose of anti-SARS-CoV-2 antibody, which is administered to a patient prior to the administration of the very next dose in the sequence with no intervening doses.

The methods, according to this aspect of the invention, may comprise administering to a patient any number of secondary and/or tertiary doses of an anti-SARS-CoV-2 antibody. For to example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In some embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

Diagnostic Uses of the Antibodies

The anti-SARS-CoV-2 antibodies of the present disclosure may be used to detect and/or measure SARS-CoV-2 in a sample, e.g., for diagnostic purposes. In some embodiments, The method may include: (i) contacting a sample with the antibody or antigen-binding fragment thereof described herein; and (ii) determining binding of the antibody or antigen-binding fragment to one or more SARS CoV-2 antigens, wherein binding of the antibody to the one or more SARS CoV-2 antigens is indicative of the presence of SARS CoV-2 in the sample.

Some embodiments may include the use of one or more antibodies of the present disclosure in assays to detect a SARS-CoV-2-associated disease or disorder. Exemplary diagnostic assays for SARS-CoV-2 may comprise, e.g., contacting a sample obtained from a patient with an anti-SARS-CoV-2 antibody of this disclosure. In some embodiments, the anti-SARS-CoV-2 antibody is labeled with a detectable label or reporter molecule or used as a capture ligand to selectively isolate SARS-CoV-2 from patient samples.

Alternatively, an unlabeled anti-SARS-CoV-2 antibody can be used in diagnostic applications in combination with a secondary antibody, which is itself detectably labeled. The detectable label or reporter molecule can be a radioisotope, such as H, C, P, S, or I; a fluorescent or chemiluminescent moiety, such as fluorescein isothiocyanate, or rhodamine; or an enzyme such as alkaline phosphatase, β-galactosidase, horseradish peroxidase, or luciferase. Specific exemplary assays that can be used to detect or measure SARS-CoV-2 in a sample include enzyme-linked immunosorbent assay (ELISA), radioimmunoassay (RIA), and fluorescence-activated cell sorting (FACS).

Samples that can be used in SARS-CoV-2 diagnostic assays include any tissue or fluid sample obtainable from a patient, which contains detectable quantities of either SARS-CoV-2 protein, or fragments thereof, under normal or pathological conditions. Generally, levels of SARS-CoV-2 protein in a particular sample obtained from a healthy patient (e.g., a patient not afflicted with a disease associated with SARS-CoV-2) will be measured to initially establish a baseline, or standard, level of SARS-CoV-2. This baseline level of SARS-CoV-2 can then be compared against the levels of SARS-CoV-2 measured in samples obtained from individuals suspected of having a SARS-CoV-2-associated condition or symptoms associated with such condition.

The antibodies specific for SARS-CoV-2 antigens may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing a N-terminal biotin will be oriented such that the C-terminal portion of the peptide will be distal to the surface.

### Combination Therapies

In yet another aspect, this disclosure additionally provides a pharmaceutical composition comprising a first isolated anti-SARS-CoV-2 antibody or antigen-binding fragment thereof described herein, and a second isolated anti-SARS-CoV-2 antibody or antigen-binding fragment thereof that binds specifically to the SARS-CoV-2 antigen.

In some embodiments, the first anti-SARS-CoV-2 antibody or antigen-binding fragment thereof and the second anti-SARS-CoV-2 antibody or antigen-binding fragment thereof bind to different epitopes of the SARS-CoV-2 antigen. In some embodiments, a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

In some embodiments, the first anti-SARS-CoV-2 antibody is an antibody as disclosed herein, e.g., Acovimab.

In some embodiments, the second anti-SARS-CoV-2 antibody comprises six CDRs (HCDR1, HCDR2, HCDR3, LCDR1, LCDR2, and LCDR3) of Sotrovimab, as set forth in SEQ ID NO: 37-42.

In some embodiments, the second anti-SARS-CoV-2 antibody comprises HCVR and/or LCVR of Sotrovimab, as set forth in SEQ ID NOs: 43 and 44.

In some embodiments, the second anti-SARS-CoV-2 antibody comprises a HCVR having at least 75% (e.g., 75%, 80%, 85%, 90%, 95%, 96%, 97%, 98%, 99%) sequence identity to the HCVR of Sotrovimab as set forth in SEQ ID NO: 43.

In some embodiments, the second anti-SARS-CoV-2 antibody comprises a LCVR having at least 75% (e.g., 75%, 80%, 85%, 90%, 95%, 96%, 97%, 98%, 99%) sequence identity to the LCVR of Sotrovimab as set forth in SEQ ID NO: 44.

In some embodiments, the second anti-SARS-CoV-2 antibody comprises Sotrovimab.

Sotrovimab (Xevudy®), also known as VIR 7831 or GSK4182136, is a recombinant human monoclonal antibody targeted against SARS-CoV-2 for the treatment of COVID-19. Sotrovimab neutralizes SARS-CoV-2 by binding to a highly conserved epitope located on the receptor binding domain (RBD) of the virus' spike protein. Preclinical studies indicate that the antibody provides a high barrier against viral escape and retains antiviral activity against newer SARS-CoV-2 variants that are associated with increased transmissibility and immune invasion, including Omicron variant. Sotrovimab is characterized in Pinto, D., et al. Nature 583, 290-295 (2020), the content of which is hereby incorporated by reference.

TABLE 2
Representative amino acid sequences of Sotrovimab

| SEQ | | |
|---|---|---|
| ID | | |
| NO | SEQUENCE | INFORMATION |
| 37 | GYPFTSYG | HCDR1 |
| 38 | ISTYNGNT | HCDR2 |
| 39 | ARDYTRGAWFGESLIGGFDN | HCDR3 |
| 40 | QTVSSTS | LCDR1 |
| 41 | GAS | LCDR2 |
| 42 | QQHDTSLT | LCDR3 |
| 43 | QVQLVQSGAEVKKPGASVKVSCKASGYPFTSYGISWVR | Sotrovimab IGHV |
| | QAPGQGLEWMGWISTYNGNTNYAQKFQGRVTMTTDTS | |
| | TTTGYMELRRLRSDDTAVYYCARDYTRGAWFGESLIGG | |
| | FDNWGQGTLVTVSS | |
| 44 | EIVLTQSPGTLSLSPGERATLSCRASQTVSSTSLAWYQQK | Sotrovimab IGLV |
| | PGQAPRLLIYGASSRATGIPDRFSGSGSGTDFTLTISRLEP | |
| | EDFAVYYCQQHDTSLTFGGGTKVEIK | |

Also provided in this disclosure is a bi-specific antibody having a first antigen binding arm having six CDRs (HCDR1, HCDR2, HCDR3, LCDR1, LCDR2, and LCDR3) of Acovidmab and a second antigen binding arm having six CDRs (HCDR1, HCDR2, HCDR3, LCDR1, LCDR2, and LCDR3) of Sotrovimab.

In some embodiments, the bi-specific antibody may include a first antigen binding arm having a HCVR of Acovidmab or having a HCVR having at least 75% (e.g., 75%, 80%, 85%, 90%, 95%, 96%, 97%, 98%, 99%) sequence identity to the HCVR of Acovidmab.

In some embodiments, the bi-specific antibody may include a first antigen binding arm having a LCVR of Acovidmab or having a LCVR having at least 75% (e.g., 75%, 80%, 85%, 90%, 95%, 96%, 97%, 98%, 99%) sequence identity to the HCVR of Acovidmab.

In some embodiments, the bi-specific antibody may include a second antigen binding arm having a HCVR of Sotrovimab or having a HCVR having at least 75% (e.g., 75%, 80%, 85%, 90%, 95%, 96%, 97%, 98%, 99%) sequence identity to the HCVR of Sotrovimab.

In some embodiments, the bi-specific antibody may include a second antigen binding arm having a LCVR of Sotrovimab or having a LCVR having at least 75% (e.g., 75%, 80%, 85%, 90%, 95%, 96%, 97%, 98%, 99%) sequence identity to the HCVR of Sotrovimab.

In another aspect, this disclosure further provides a method of neutralizing SARS-CoV-2 or treating a SARS-CoV-2 infection in a subject. In some embodiments, the method comprising administering to a subject in need thereof a therapeutically effective amount of a first isolated anti-SARS-CoV-2 antibody or antigen-binding fragment thereof described herein, and a therapeutically effective amount of a second isolated anti-SARS-CoV-2 antibody or antigen-binding fragment thereof that binds specifically to the SARS-CoV-2 antigen.

In some embodiments, the first anti-SARS-CoV-2 antibody or antigen-binding fragment thereof and the second anti-SARS-CoV-2 antibody or antigen-binding fragment thereof bind to different epitopes of the SARS-CoV-2 antigen. In some embodiments, a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

In some embodiments, the first anti-SARS-CoV-2 antibody is an antibody as disclosed herein, e.g., Acovimab.

In some embodiments, the second anti-SARS-CoV-2 antibody comprises six CDRs (HCDR1, HCDR2, HCDR3, LCDR1, LCDR2, and LCDR3) of Sotrovimab, as set forth in SEQ ID NO: 37-42.

In some embodiments, the second anti-SARS-CoV-2 antibody comprises HCVR and/or LCVR of Sotrovimab, as set forth in SEQ ID NOs: 43 and 44.

In some embodiments, the second anti-SARS-CoV-2 antibody comprises a HCVR having at least 75% (e.g., 75%, 80%, 85%, 90%, 95%, 96%, 97%, 98%, 99%) sequence identity to the HCVR of Sotrovimab as set forth in SEQ ID NO: 43.

In some embodiments, the second anti-SARS-CoV-2 antibody comprises a LCVR having at least 75% (e.g., 75%, 80%, 85%, 90%, 95%, 96%, 97%, 98%, 99%) sequence identity to the LCVR of Sotrovimab as set forth in SEQ ID NO: 44.

In some embodiments, the second anti-SARS-CoV-2 antibody comprises Sotrovimab.

Combination therapies may include an anti-SARS-CoV-2 antibody of the invention and any additional therapeutic agent that may be advantageously combined with an antibody of this disclosure or with a biologically active fragment thereof. The antibody may be combined synergistically with one or more drugs or therapy used to treat a disease or disorder associated with a viral infection, such as a SARS-CoV-2 infection. In some embodiments, the antibody may be combined with a second therapeutic agent to ameliorate one or more symptoms of the disease. In some embodiments, the antibody may be combined with a second antibody to provide synergistic activity in ameliorating one or more symptoms of the disease. In some embodiments, the first antibody or antigen-binding fragment thereof is administered before, after, or concurrently with administration of the second therapeutic agent.

As used herein, the term “in combination with” means that additional therapeutically active component(s) may be administered prior to, concurrent with, or after the administration of the anti-SARS-CoV-2 antibody of the present disclosure. The term “in combination with” also includes sequential or concomitant administration of an anti-SARS-CoV-2 antibody and a second therapeutic agent.

The additional therapeutically active component(s) may be administered to a subject prior to administration of an anti-SARS-CoV-2 antibody of the present disclosure. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-SARS-CoV-2 antibody of the present disclosure. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-SARS-CoV-2 antibody of the present disclosure. “Concurrent” administration, for purposes of the present disclosure, includes, e.g., administration of an anti-SARS-CoV-2 antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-SARS-CoV-2 antibody and the additional therapeutically active component may be administered intravenously, etc.); alternatively, each dosage form may be administered via a different route (e.g., the anti-SARS-CoV-2 antibody may be administered intravenously, and the additional therapeutically active component may be administered orally). In any event, administering the components in a single dosage form, in separate dosage forms by the same route, or in separate dosage forms by different routes are all considered “concurrent administration,” for purposes of the present disclosure. For purposes of the present disclosure, administration of an anti-SARS-CoV-2 antibody “prior to,” “concurrent with,” or “after” (as those terms are defined hereinabove) administration of an additional therapeutically active component is considered administration of an anti-SARS-CoV-2 antibody “in combination with” an additional therapeutically active component.

In some embodiments, the method comprises administering to a subject a first anti-SARS-CoV-2 antibody or antigen-binding fragment thereof and/or a second anti-SARS-CoV-2 antibody or antigen-binding fragment thereof at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, multiple doses of a first anti-SARS-CoV-2 antibody or antigen-binding fragment thereof in combination with a second anti-SARS-CoV-2 antibody or antigen-binding fragment thereof may be administered to a subject over a defined time course. In some embodiments, the method comprises sequentially administering to a subject one or more doses of a first anti-SARS-CoV-2 antibody or antigen-binding fragment thereof in combination with one or more doses of a second anti-SARS-CoV-2 antibody or antigen-binding fragment thereof.

As used herein, “sequentially administering” means that each dose of the antibody is administered to the subject at a different point in time, e.g., on different days separated by a predetermined interval (e.g., hours, days, weeks or months). The present invention includes methods that comprise sequentially administering to the patient a single initial dose of an anti-SARS-CoV-2 antibody or antigen-binding fragment thereof, followed by one or more secondary doses of the anti-SARS-CoV-2 antibody or antigen-binding fragment thereof, and optionally followed by one or more tertiary doses of the anti-SARS-CoV-2 antibody or antigen-binding fragment thereof. In some embodiments, the methods further comprise sequentially administering to the patient a single initial dose of, followed by one or more secondary doses of an anti-SARS-CoV-2 antibody or antigen-binding fragment thereof, and optionally followed by one or more tertiary doses of the anti-SARS-CoV-2 antibody or antigen-binding fragment thereof.

In some embodiments, multiple doses of a first anti-SARS-CoV-2 antibody or antigen-binding fragment thereof and a second anti-SARS-CoV-2 antibody or antigen-binding fragment thereof may be administered to a subject over a defined time course. The methods according to this aspect of the invention comprise sequentially administering to a subject multiple doses of the first anti-SARS-CoV-2 antibody or antigen-binding fragment thereof and the second anti-SARS-CoV-2 antibody or antigen-binding fragment thereof. As used herein, “sequentially administering” means that each dose of the first anti-SARS-CoV-2 antibody or antigen-binding fragment thereof in combination with the second anti-SARS-CoV-2 antibody or antigen-binding fragment thereof is administered to the subject at a different point in time, e.g., on different days separated by a predetermined interval (e.g., hours, days, weeks or months).

In some embodiments, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, a dose of a first anti-SARS-CoV-2 antibody or antigen-binding fragment thereof (and/or a second anti-SARS-CoV-2 antibody or antigen-binding fragment thereof) which is administered to a patient prior to the administration of the very next dose in the sequence with no intervening doses.

In some embodiments, the methods may include administering to a patient any number of secondary and/or tertiary doses of a first anti-SARS-CoV-2 antibody or antigen-binding fragment thereof (and/or a second anti-SARS-CoV-2 antibody or antigen-binding fragment thereof). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, one or more doses of a first anti-SARS-CoV-2 antibody or antigen-binding fragment thereof (and/or a second anti-SARS-CoV-2 antibody or antigen-binding fragment thereof) antibody or antigen-binding fragment thereof and/or an anti-SARS-CoV-2 antibody or antigen-binding fragment thereof are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

In the case of the first or second anti-SARS-CoV-2 antibody or antigen-binding fragment thereof, a therapeutically effective amount can be from about 0.02 mg, about 0.05 mg, about 0.1 mg, about 0.5 mg, about 1 mg, about 5 mg, about 10 mg, about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 660 mg, about 680 mg, about 700 mg, 720 mg, about 740 mg, about 760 mg, about 780 mg, about 760 mg, about 780 mg, about 800 mg, 820 mg, about 840 mg, about 860 mg, about 880 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, about 1200 mg of the anti-SARS-CoV-2 antibody or antigen-binding fragment thereof.

The amount of either the first or the second anti-SARS-CoV-2 antibody or antigen-binding fragment thereof contained within the individual doses may be expressed in terms of milligrams of antibody per kilogram of subject body weight (i.e., mg/kg or mpk). In some embodiments, either the first or the second anti-SARS-CoV-2 antibody or antigen-binding fragment thereof used in the methods of this disclosure may be administered to a subject at a dose of about 0.0001 to about 100 mg/kg of subject body weight. For example, an anti-SARS-CoV-2 antibody or antigen-binding fragment thereof may be administered at a dose of about 0.1 mg/kg to about 20 mg/kg of a patient's body weight.

### Chimeric Immunogens

Polypeptides

In another aspect, this disclosure provides an isolated polypeptide comprising an amino acid sequence having at least 75% (i.e., 75%, 80%, 85%, 90%, 95%, 97%, 98%, 99% or more) sequence identity to the amino acid sequence of SEQ ID NO: 13 or having the amino acid sequence of SEQ ID NO: 13.

“Polypeptide” is used in its conventional meaning, i.e., as a sequence of amino acids. The polypeptides are not limited to a specific length of the product. Peptides, polypeptides, and proteins are included within the definition of the polypeptide, and such terms can be used interchangeably herein unless specifically indicated otherwise. This term also includes post-expression modifications of the polypeptide, for example, glycosylations, acetylations, phosphorylations, and the like, as well as other modifications known in the art, both naturally occurring and non-naturally occurring. A polypeptide can be an entire protein or a subsequence thereof. A polypeptide “variant,” as the term is used herein, is a polypeptide that typically differs from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of some techniques well known in the art.

For example, certain amino acids can be substituted for other amino acids in a protein structure without appreciable loss of its ability to bind other polypeptides (for example, antigens) or cells. Since it is the binding capacity and nature of a protein that defines that protein's biological functional activity, certain amino acid sequence substitutions can be made in a protein sequence, and, accordingly, its underlying DNA coding sequence, whereby a protein with like properties is obtained. It is thus contemplated that various changes can be made in the peptide sequences of the disclosed compositions, or corresponding DNA sequences that encode said peptides without appreciable loss of their biological utility or activity.

Variant sequences include those wherein conservative substitutions have been introduced by modification of polynucleotides encoding polypeptides of this disclosure. Amino acids can be classified according to physical properties and contribution to secondary and tertiary protein structure. Such conservative modifications include amino acid substitutions, additions, and deletions. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

“Sequence identity” or “homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least about 70%, at least about 75%, at least about 80%, at least about 90%, at least about 95%, at least about 98%, or at least about 99% polynucleotide or polypeptide homology with a polynucleotide or polypeptide described herein.

Polypeptide variant sequences may share 70% or more (i.e. 80%, 85%, 90%, 95%, 97%, 98%, 99% or more) sequence identity with the sequences recited in this disclosure. Polypeptide variants may also include polypeptide fragments comprising various lengths of contiguous stretches of amino acid sequences disclosed herein. Polypeptide variant sequences include at least about 5, 10, 15, 20, 30, 40, 50, 75, 100, 150, or more contiguous peptides of one or more of the sequences disclosed herein as well as all intermediate lengths therebetween.

Nucleic Acids

Also within the scope of this disclosure are a nucleic acid molecule encoding the polypeptide, a vector comprising the nucleic acid molecule, and a host cell comprising the nucleic acid or the vector, as described herein.

Further provided in this disclosure is a virus-like particle comprising the polypeptide, the nucleic acid, or the vector, as described herein.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded.

An “isolated nucleic acid” refers to a nucleic acid, the structure of which is not identical to that of any naturally occurring nucleic acid or to that of any fragment of a naturally occurring genomic nucleic acid. The term, therefore, covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide, fusion protein, or antibody of this invention. For this purpose, one can operatively link the nucleic acid to suitable regulatory sequences to generate an expression vector.

The nucleic acid and amino acid sequences disclosed herein are shown using standard letter abbreviations for nucleotide bases, and one letter code for amino acids. Only one strand of each nucleic acid sequence is shown, but the complementary strand is understood as included by any reference to the displayed strand.

This disclosure also includes vectors containing a coding sequence for the disclosed immunogen, host cells containing the vectors, and methods of making substantially pure immunogen comprising the steps of introducing the coding sequence for the immunogen into a host cell, and cultivating the host cell under appropriate conditions such that the immunogen is produced and secreted. The immunogen so produced may be harvested in conventional ways. Therefore, the present invention also relates to methods of expressing the immunogen and biological equivalents disclosed herein, assays employing these gene products, and recombinant host cells which comprise DNA constructs which express these receptor proteins.

The disclosed immunogens may be recombinantly expressed by molecular cloning the nucleic acid encoding the immunogens into an expression vector (such as pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce the immunogens. Techniques for such manipulations can be found described in Sambrook et al., 1989, Molecular Cloning: A Laboratory Manual; Cold Spring Harbor Laboratory, Cold Spring Harbor, New York, are well known and readily available to the artisan of ordinary skill in the art. Therefore, another aspect of the present invention includes host cells that have been engineered to contain and/or express DNA sequences encoding the immunogens. Such recombinant host cells can be cultured under suitable conditions to produce the disclosed immunogens or a biologically equivalent form. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines.

For instance, one insect expression system utilizes Spodoptera frugiperda (Sf21) insect cells (Invitrogen) in tandem with a baculovirus expression vector (pAcG2T, Pharmingen). Also, mammalian species which may be suitable and which are commercially available, include but are not limited to, L cells L-M(TK˜) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

A variety of mammalian expression vectors may be used to express recombinant immunogens in mammalian cells. Expression vectors are defined herein as DNA sequences that are required for the transcription of cloned DNA and the translation of their mRNAs in an appropriate host. Such vectors can be used to express eukaryotic DNA in a variety of hosts, such as bacteria, blue-green algae, plant cells, insect cells, and animal cells. Specifically designed vectors allow the shuttling of DNA between hosts such as bacteria-yeast or bacteria-animal cells. An appropriately constructed expression vector should contain: an oπgm of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency.

Expression vectors may include, but are not limited to, cloning vectors, modified cloning vectors, specifically designed plasmids or viruses. Commercially available mammalian expression vectors which may be suitable for immunogen expression, include but are not limited to, pIRES-hyg (Clontech), pIRES-puro (Clontech), pcDNA3.neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAlamp (Invitrogen), pcDNA3 (Invitrogen), pMClneo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

Also, a variety of bacterial expression vectors may be used to express the disclosed immunogens in bacterial cells. Commercially available bacterial expression vectors that may be suitable for immunogen expression include, but are not limited to pCR2.1 (Invitrogen), pETl la (Novagen), lambda gtl 1 (Invitrogen), and pKK223-3 (Pharmacia).

In addition, a variety of fungal cell expression vectors may be used to express the immunogens in fungal cells. Commercially available fungal cell expression vectors which may be suitable for recombinant immunogen expression include but are not limited to pYES2 (Invitrogen) and Pichia expression vector (Invitrogen).

Also, a variety of insect cell expression vectors may be used to express a recombinant receptor in insect cells. Commercially available insect cell expression vectors which may be suitable for recombinant expression of the immunogens include but are not limited to pBlueBacIII and pBlueBacHιs2 (Invitrogen), and pAcG2T (Pharmingen).

The expression vector may be introduced into host cells via any one of a number of techniques, including but not limited to, transformation, transfection, protoplast fusion, and electroporation. Transformation is meant to encompass a genetic change to the target cell resulting from incorporation of DNA. Transfection is meant to include any method known in the art for introducing the immunogens into the test cells. For example, transfection includes calcium phosphate or calcium chloride mediated transfection, lipofection, electroporation, as well as infection with, for example, a viral vector such as a recombinant retroviral vector containing the nucleotide sequence which encodes the immunogens, and combinations thereof. The expression vector-containing cells are individually analyzed to determine whether they produce the immunogens. Identification of immunogen expressing cells may be done by several means, including but not limited to immunological reactivity with specific bNAbs, labeled ligand binding and the presence of host cell-associated activity with respect to the immunogens.

Also within the scope of this invention is a host cell that contains the above-described nucleic acid. Examples include bacterial cells (e.g., E. coli cells, insect cells (e.g., using baculovirus expression vectors), yeast cells, or mammalian cells. See, e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

As used herein, the term “virus-like particle” (VLP) refers to a structure resembling a virus but which has not been demonstrated to be pathogenic. Typically, a virus-like particle in accordance with the disclosure does not carry genetic information encoding for the proteins of the virus-like particle. In general, virus-like particles lack the viral genome and, therefore, are noninfectious. Also, virus-like particles can often be produced in large quantities by heterologous expression and can be easily purified. Some virus-like particles may contain nucleic acid distinct from their genome. In some embodiments, a virus-like particle may be non-replicative and noninfectious since it lacks all or part of the viral genome, in particular, the replicative and infectious components of the viral genome. A virus-like particle may contain nucleic acid distinct from its genome. In some embodiments, a virus-like particle is a viral capsid, such as the viral capsid of the corresponding virus, bacteriophage, or RNA phage. The terms “viral capsid” or “capsid,” as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA phages or HBcAg's have a spherical form of icosahedral symmetry. The term “capsid-like structure,” as used herein, refers to a macromolecular assembly composed of viral protein subunits resembling the capsid morphology in the above-defined sense but deviating from the typical symmetrical assembly while maintaining a sufficient degree of order and repetitiveness.

Immunogenic Compositions

In another aspect, this disclosure provides an immunogenic composition for stimulating an immune response in a subject in need thereof. The immunogenic composition includes (i) the immunogen, the nucleic acid, the host cell, or the virus particle described above; and (ii) a pharmaceutically acceptable carrier.

In another aspect, this disclosure provides an immunogenic composition for stimulating an immune response in a subject in need thereof. In some embodiments, the immunogenic composition comprises the polypeptide, the nucleic acid, the vector, the virus-like particle, as described herein, or a combination thereof.

In some embodiments, the immunogenic composition is capable of inducing production of at least two anti-SARS-CoV2 antibodies that bind specifically to different epitopes on a spike protein of a SARS-CoV2 variant.

Methods of Use of Chimeric Immunogens

In another aspect, this disclosure provides a method of stimulating an immune response in a subject in need thereof. In some embodiments, the method comprises administrating to the subject an effective amount of the polypeptide, the nucleic acid, the vector, the virus-like particle, the immunogenic composition, as described herein, or a combination thereof.

In another aspect, this disclosure provides a method of neutralizing SARS-CoV-2 in a subject in need thereof. In some embodiments, the method comprises administrating to the subject an effective amount of the polypeptide, the nucleic acid, the vector, the virus-like particle, the immunogenic composition, as described herein, or a combination thereof.

In another aspect, this disclosure provides a method of treating a SARS-CoV-2 infection in a subject in need thereof. In some embodiments, the method comprises administrating to the subject an effective amount of the polypeptide, the nucleic acid, the vector, the virus-like particle, the immunogenic composition, as described herein, or a combination thereof.

The immunogens, as disclosed herein, a nucleic acid molecule encoding the disclosed immunogen, the host cell, or the virus particle, can be administered to a subject in order to generate an immune response to a pathogen, SARS-CoV-2. In another aspect, this disclosure provides a method of treating or preventing SARS-COV-2 infection in a subject in need thereof. The method includes administering to the subject a therapeutically effective amount of the immunogen, the nucleic acid, the host cell, the protein complex, or the virus particle described above, or a combination thereof. This disclosure also provides use of the immunogen, the nucleic acid, the host cell, the protein complex, or the virus particle described above, or a combination thereof in the preparation of a medicament to treat or prevent SARS-COV-2 infection in a subject.

In exemplary applications, compositions are administered to a subject suffering from SARS-COV-2 infection or at risk of becoming infected by SARS-COV-2. In other applications, the immunogens disclosed herein can be administered prophylactically, for example, as part of an immunization regimen.

The immunogen is administered in an amount sufficient to raise an immune response against the SARS-COV-2 virus. Administration induces a sufficient immune response to treat the pathogenic infection, for example, to inhibit the infection and/or reduce the signs and/or symptoms of the infection. Amounts effective for this use will depend upon the severity of the disease, the general state of the subject's health, and the robustness of the subject's immune system. A therapeutically effective amount of the immunogen is that which provides either subjective relief of a symptom(s) or an objectively identifiable improvement as noted by the clinician or other qualified observers.

A therapeutically effective amount or effective amount refers to the amount of agents, such as a nucleic acid vaccine or other therapeutic agents, that is sufficient to prevent, treat (including prophylaxis), reduce and/or ameliorate the symptoms and/or underlying causes of any of a disorder or disease, for example, to prevent, inhibit, and/or treat SARS-COV-2. In some embodiments, an “effective amount” is sufficient to reduce or eliminate a symptom of a disease, such as AIDS. For instance, this can be the amount necessary to inhibit viral replication or to measurably alter outward symptoms of the viral infection. In general, this amount will be sufficient to measurably inhibit virus (for example, SARS-CoV-2) replication or infectivity. When administered to a subject, a dosage will generally be used that will achieve target tissue concentrations (for example, in lymphocytes) that have been shown to achieve in vitro inhibition of viral replication.

An immunogen can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, PA, 1995) either locally or systemically, such as by intramuscular, subcutaneous, or intravenous injection, but even oral, nasal, or anal administration is contemplated. In one embodiment, the administration is by subcutaneous or intramuscular injection. To extend the time during which the disclosed immunogen is available to stimulate a response, the immunogen can be provided as an implant, an oily injection, or as a particulate system. The particulate system can be a microparticle, a microcapsule, a microsphere, a nanocapsule, or a similar particle (see, e.g., Banga, supra). A particulate carrier based on a synthetic polymer has been shown to act as an adjuvant to enhance the immune response, in addition to providing a controlled release. Aluminum salts can also be used as adjuvants to produce an immune response.

Optionally, one or more cytokines, such as interleukin (IL)-2, IL-6, IL-12, IL-15, RANTES, granulocyte-macrophage colony-stimulating factor (GM-CSF), tumor necrosis factor (TNF)-a, interferon (IFN)-a or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2): 122-38; Lotze et al., 2000, Cancer J Sci. Am. 6(Suppl 1):S61-6; Cao et al., 1998, Stem Cells 16(Suppl 1 J.-251-60; Kuiper et al., 2000, Adv. Exp. Med. Biol. 465:381-90). These molecules can be administered systemically (or locally) to the host. In several examples, IL-2, RANTES, GM-CSF, TNF-α, IFN-γ, G-CSF, LFA-3, CD72, B7-1, B7-2, B7-1 B.7-2, OX-40L, 41 BBL, and ICAM-1 are administered.

A pharmaceutical composition including an isolated immunogen is provided. In some embodiments, the immunogen is mixed with an adjuvant containing two or more of a stabilizing detergent, a micelle-forming agent, and an oil. Suitable stabilizing detergents, micelle-forming agents, and oils are detailed in U.S. Pat. Nos. 5,585,103; 5,709,860; 5,270,202; and 5,695,770. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, DE), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle-forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54: 110, 1977, and Hunter et al., J. Immunol 129: 1244, 1981, PLURONIC™ L62LF, L101, and L64, PEG1000, and TETRONIC™ 1501, 150R1, 701, 901, 1301, and 130R1. The chemical structures of such agents are well-known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example, between 0.5 and 10%, or in an amount between 1.25 and 5%.

Controlled-release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, PA, 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm, so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, NY, pp. 219-342, 1994; Tice & Tabibi, Treatise on Controlled Drug Delivery, A. Kydonieus, ed., Marcel Dekker, Inc. New York, NY, pp. 315-339, 1992).

Polymers can be used for ion-controlled release. Various degradable and nondegradable polymeric matrices for use in controlled drug delivery are known in the art (Langer, Accounts Chem. Res. 26:53', 1993). For example, the block copolymer, poloxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, /. Parent. Sci. Tech. 44(2):58, 1990). Alternatively, hydroxyapatite has been used as a microcarrier for controlled release of proteins (Ijntema et ah, Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA, 1993). Numerous additional systems for controlled delivery of therapeutic proteins are known (e.g., U.S. Pat. Nos. 5,055,303; 5,188,837; 4,235,871; 4,501,728; 4,837,028; 4,957,735; and 5,019,369; 5,055,303; 5,514,670; 5,413,797; 5,268,164; 5,004,697; 4,902,505; 5,506,206; 5,271,961; 5,254,342; and 5,534,496).

In another embodiment, a pharmaceutical composition includes a nucleic acid encoding a disclosed immunogen. A therapeutically effective amount of the nucleic acid can be administered to a subject in order to generate an immune response. In one specific, non-limiting example, a therapeutically effective amount of a nucleic acid encoding a disclosed gp120 immunogen or immunogenic fragment thereof is administered to a subject to treat or prevent or inhibit SARS-COV-2 infection.

Optionally, one or more cytokines, such as IL-2, IL-6, IL-12, RANTES, GM-CSF, TNF-α, or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2): 122-38; Lotze et al., 2000, Cancer J Sci. Am. 6(Suppl 1): S61-6; Cao et al., 1998, Stem Cells 16(Suppl 1):251-60; Kuiper et al., 2000, Adv. Exp. Med. Biol. 465:381-90). These molecules can be administered systemically to the host. It should be noted that these molecules can be co-administered via insertion of a nucleic acid encoding the molecules into a vector, for example, a recombinant pox vector (see, for example, U.S. Pat. No. 6,045,802). In various embodiments, the nucleic acid encoding the biological adjuvant can be cloned into the same vector as the disclosed immunogen coding sequence, or the nucleic acid can be cloned into one or more separate vectors for co-administration. In addition, nonspecific immunomodulating factors such as Bacillus Cahnette-Guerin (BCG) and levamisole can be co-administered. One approach to administration of nucleic acids is direct immunization with plasmid DNA, such as with a mammalian expression plasmid. As described above, the nucleotide sequence encoding the disclosed immunogen can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired immunogen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a disclosed immunogen can also be expressed by attenuated viral hosts or vectors or bacterial vectors. Recombinant vaccinia virus, adeno-associated virus (AAV), herpes virus, retrovirus, cytomegalovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed immunogen is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

It may be advantageous to administer the immunogenic compositions disclosed herein with other agents such as proteins, peptides, antibodies, and other antiviral agents, such as anti-SARS-COV-2 agents. In certain embodiments, the immunogenic compositions are administered sequentially with other anti-HIV therapeutic agents, such as before or after the other agent. One of ordinary skill in the art would know that sequential administration can mean immediately following or after an appropriate period of time, such as hours, days, weeks, months, or even years later.

One can also use cocktails containing the disclosed immunogenic agents, for example, the immunogen, the nucleic acid encoding the immunogen, the host cell, or the virus particle described above, or a combination thereof to prime and then boost with trimers from a variety of different HIV strains or with trimers that are a mixture of multiple HIV strains. The prime can be administered as a single dose or multiple doses, for example, two doses, three doses, four doses, five doses, six doses or more can be administered to a subject over days, weeks or months. The boost can be administered as a single dose or multiple doses, for example, two to six doses or more can be administered to a subject over a day, a week or months. Multiple boosts can also be given, such as one to five, or more. Different dosages can be used in a series of sequential inoculations. For example, a relatively large dose in a primary inoculation and then a boost with relatively smaller doses. The immune response against the selected antigenic surface can be generated by one or more inoculations of a subject with an immunogenic composition disclosed herein.

For in vitro use, an immunogenic composition may consist of the isolated protein, peptide epitope, or nucleic acid encoding the protein or peptide epitope. For in vivo use, the immunogenic composition will typically include the protein, immunogenic peptide or nucleic acid in pharmaceutically acceptable carriers and/or other agents. Any particular peptide, such as a disclosed immunogen or a nucleic acid encoding the immunogen, can be readily tested for its ability to induce a B cell response by art-recognized assays. Immunogenic compositions can include adjuvants, which are well known to one of skill in the art.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically-acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, TWEENS or SPANS or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally comprise one or more excipients or diluents. In some preferred embodiments, the topical formulations include a material that would enhance absorption or penetration of the active agent(s) through the skin or other affected areas. The topical composition is useful for treating inflammatory disorders in the skin, including, but not limited to, eczema, acne, rosacea, psoriasis, contact dermatitis, and reactions to poison ivy.

A topical composition contains a safe and effective amount of a dermatologically acceptable carrier suitable for application to the skin. A “cosmetically acceptable” or “dermatologically-acceptable” composition or component refers to a composition or component that is suitable for use in contact with human skin without undue toxicity, incompatibility, instability, allergic response, and the like. The carrier enables an active agent and an optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular, one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition. The topical composition may be a cosmetic or dermatologic product in the form known in the art for topical or transdermal applications, including solutions, aerosols, creams, gels, patches, ointment, lotion, or foam.

Pharmaceutically acceptable carrier includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19).

The host cells provided in the immunogenic compositions may be inactivated or chemically/genetically attenuated bacterial vaccine that does not elicit the cytotoxic T-lymphocyte (CTL) immune response necessary for the lysis of tumor cells and cells infected with intracellular pathogens.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

A “neutralizing antibody” is one that can neutralize the ability of that pathogen to initiate and/or perpetuate an infection in a host and/or in target cells in vitro. “Broadly neutralizing anti-SARS-CoV-2 antibodies” refer to antibodies that neutralize more than one SARS-CoV-2 virus strains/variants in a neutralization assay. A broad neutralizing anti-SARS-CoV-2 antibody may neutralize at least 2, 3, 4, 5, 6, 7, 8, 9 or more different strains/variants of SARS-CoV-2.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen (e.g., a spike or S protein of SARS-CoV-2 virus). It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the VL, VH, CL, and CHI domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3rd ed. 1993)); (iv) a Fd fragment consisting of the VH and CHI domains; (v) a Fv fragment consisting of the VL and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds to a spike or S protein of SARS-CoV-2 virus is substantially free of antibodies that specifically bind antigens other than the neuraminidase). An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody), and the constant region sequence or Fc is derived from a different source (e.g., a different antibody, such as an IgG, IgA2, IgD, IgE or IgM antibody).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that least two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

The term “specifically binds,” or “binds specifically to,” or the like, refers to an antibody that binds to a single epitope, e.g., under physiologic conditions, but which does not bind to more than one epitope. Accordingly, an antibody that specifically binds to a polypeptide will bind to an epitope that is present on the polypeptide, but which is not present on other polypeptides. Specific binding can be characterized by an equilibrium dissociation constant of at least about 1×10-8 M or less (e.g., a smaller KD denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to a spike or S protein of a SARS-CoV-2 virus.

For example, the antibody binds to a spike or S protein with “high affinity,” namely with a KD of 1×10-7 M or less, more preferably 5×10-8 M or less, more preferably 3×10-8 M or less, more preferably 1×10-8 M or less, more preferably 5×10-9 M or less or even more preferably 1×10-9 M or less, as determined by surface plasmon resonance, e.g., BIACORE. The term “does not substantially bind” to a protein or cells, as used herein, means does not bind or does not bind with a high affinity to the protein or cells, i.e., binds to the protein or cells with a KD of 1×10-6 M or more, more preferably 1×10-5 M or more, more preferably 1×10-4 M or more, more preferably 1×10-3 M or more, even more preferably 1×10-2 M or more.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a BIACORE system.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

The term “eliciting” or “enhancing” in the context of an immune response refers to triggering or increasing an immune response, such as an increase in the ability of immune cells to kill pathogens (e.g., SARS-CoV-2) and pathogen-infected cells.

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent;

surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of this disclosure, and are physiologically acceptable to the subject. Supplementary active compounds may also be incorporated into the compositions.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials, methods, and instrumentation used in Example 2.

Single CoV2-RBD^{WT+}RBD^{BA.1+} memory B cells were sorted from COVID-19 vaccinated CoV2-infected individuals and proliferated in vitro in 96 well plates for 2-3 weeks to induce further somatic hypermutation in IGVH and IGVL genes. Supernatants of the proliferated cells were screened for binding breadth for different CoV2 variant RBD and potent neutralization against CoV2-WT and Omicron, and positive wells were selected and used to isolate monoclonal antibodies. This approach allowed for isolating a novel recombinant monoclonal human antibody that possessed potent neutralizing activity against the parental CoV2 isolate and earlier (Alpha, Beta, Gamma, and Delta) variants and was also able to potently neutralize the BA.1, BA.2, BA.2.12.1, BA.2.75, BA.4/5 and BA4.6 subvariants of currently circulating CoV2 Omicron strain.

### Antigens Used

RBD-WT antigens were expressed as fusion proteins with the gp70 carrier domain as previously described (Datta P, et al. J Immunol Methods. 2021; 499:113165). In brief, a gene fragment of the CoV2-Spike gene encoding the RBD was synthesized commercially (Integrated DNA Technologies, Coralville, IA) and cloned at the 3′ end of a gene expressing the N-terminal fragment of the Friend ectotropic MuLV (Fr-MuLV) surface protein (SU) gp70 gene in the expression vector pcDNA3.4 (Addgene, Watertown, MA). The resulting plasmid was transfected into 293F cells using the Expi293 Expression System (Thermo Fisher Scientific) according to the manufacturer's protocol. Commercial preparations of the Omicron RBD (Cat #SPD-0522f), Omicron 51 (Cat #123456), Omicron Spike (Cat #123456), CoV2-D614G S1 (Cat #123456), and CoV2-D614G Spike were purchased from Acro Biosystems.

### Antigen-Antibody Binding Detected by Enzyme-Linked Immunosorbent Assay (ELISA).

RBD plasma antibodies were detected using RBD-WT protein-binding ELISA, and recombinant RBD, 51, and spike proteins from CoV2-WT and Omicron BA.1 variants were used to detect memory B cell culture supernatants and for mAb binding studies. In brief, different proteins were coated overnight at 4° C. at a concentration of 100 ng/well in 50 μl of bicarbonate buffer (pH=9.8) using U-shaped medium binding 96-well ELISA plates (Greiner Bio-One; Cat #:650001). Non-fat dry milk (2%) was used to block the ELISA plate and prepare plasma, culture supernatant, and mAb dilutions. Serially diluted plasma, culture supernatant, and mAbs were incubated at 50 μl/well in duplicates for one hour at 37° C. in a blocked plate after washing with wash buffer (1×PBS+0.1% Tween 20), and the antigen-antibody binding was detected by Alkaline phosphatase-conjugated goat anti-human IgG detector antibodies (Jackson ImmunoResearch Laboratories, West Grove, Pa) using 50 μl/well diethanolamine (DAE) buffer.

CoV2 Pseudovirus (psV) Preparation and the ACE2-HeLa Cell-Based Neutralization Assay.

Codon-optimized D614G, alpha, beta, gamma, delta, kappa, lambda, and omicron spike gene sequences with 18 amino acid C-terminal truncations were cloned into the pCAGGS vector (Ferrara F, et al. Nat Commun. 2022; 13(1):462). Additional Omicron variant spike protein-expressing plasmids were obtained from the Burton lab at the Scripps Institute. Different CoV2 pseudovirus variants were generated by co-transfecting spikes and pnl4.3. Luc. r-e-plasmids into HEK 293T cells to produce the CoV2 psV as described earlier and HuACE2-HeLa cells were infected with the CoV2 psV in DMEM media supplemented with polybrene (10 μg/ml), and infectivity determined by measuring luciferase activity at 72 h post-infection in cell lysates using luciferase substrate (Britelite, PE). To measure the neutralization potency of plasma, memory B cell culture supernatant, and mAb, psV dilutions yielding ˜100,000 RLU were used to infect Hu-ACE2-HeLa cells in the presence of titrated plasma, culture supernatant, and mAbs.

Preparation of CNBr-Sepharose Bead Columns with Immobilized RBD for the Absorption of Plasma RBD Antibodies.

Cyanogen bromide (CNBr)-activated Sepharose 4 B(Pharmacia #17-0430-01) beads were hydrated with 1 mM HCl for 30 min at room temperature, washed with excess 1×PBS and incubated overnight with the RBD protein in 1×PBS at 4° C. on a vertical rotator. After washing with excess 1×PBS, the RBD-conjugated beads were blocked with 1M ethanolamine (pH 8.0) for two hours at room temperature on a vertical rotator, after which the beads were stored in a refrigerator at 4° C. for plasma RBD antibody absorption experiments. For absorption, 1,000 μl of 10-fold diluted plasma from selected patients were incubated with the beads in a tube 0/N at 4° C. on a vertical rotator, and the absorbed plasma samples were spun at 300×g to collect the supernatant. Absorbed and unabsorbed plasma at the same dilutions was tested for RBD-WT binding by ELISA to determine the percentage of absorption, and antibody titers against an N-terminal domain (NTD) were used to measure the extent of nonspecific changes in plasma antibody concentrations. RBD-absorbed and -unabsorbed plasma was stored at −80° C. and used for the neutralization experiments. Subjects with a high RBD-specific plasma antibody neutralization breadth and potency against CoV2 variants were selected for isolation of broadly neutralizing anti-RBD antibodies.

RBD^{WT+} and RBD^{BA.1+} Bound Memory B Cell Sorting.

Two vials of cryopreserved PBMCs with cell counts of 5 million/vial were recovered from the liquid N2 storage, thawed at 37° C., and washed with 10% AMEM cell culture medium. Cells were treated with the Fc-block for 5 min at room temperature (RT), followed by incubation at 4° C. for 30 min with BUV395 tagged mouse anti-human CD3⁺, CD4⁺, CD8⁺, and CD14⁺ antibodies. After 30 min of incubation, CoV2^{WT}RBD^{AF647}, Omicron-BA.1RBD^{AF592}, anti-human CD19^{PE/Cy7} and IgG^{FITC} antibodies of mouse origin were added to the cells, and the tubes were incubated for an additional 30 min in a cold room over the nutator. The total volume of the cell suspension was maintained at <500 μl. After a second incubation at 4° C., the cells were washed three times with 1×PBS supplemented with 2% FBS and resuspended in 500 μL of 2% AMEM media, and CD19^{PE/Cy7+}IgG^{FITC+}CoV2^{WT}RBD^{AF647+}BA.1-RBD^{AF592+} single memory B cells were sorted into flat-bottom culture 96-well plates pre-plated with MS40L cells for in vitro proliferation in the presence of IL2, IL4, IL10, IL21, and CpG.

### Generation of Recombinant Broadly CoV2 Neutralizing Monoclonal Antibodies.

Cells from wells with anti-RBD antibody binding and neutralizing activity were lysed to prepare cDNA, following the protocol provided by the Invitrogen SuperScript IV First-Strand Synthesis System. Immunoglobulin heavy chain (IGVH) and immunoglobulin light chain (IGVL) were amplified using oligos described earlier (Scheid J F, et al. Science. 2011; 333(6049):1633-1637), and the antibody genes were cloned into the respective human IgG1 heavy and kappa light chain expressing vectors to produce functional human anti-CoV2 mAb.

### Example 2

The Majority of Omicron-Neutralizing Plasma Activity Targets RBD Epitopes Conserved Between CoV2WT and Omicron BA.1.

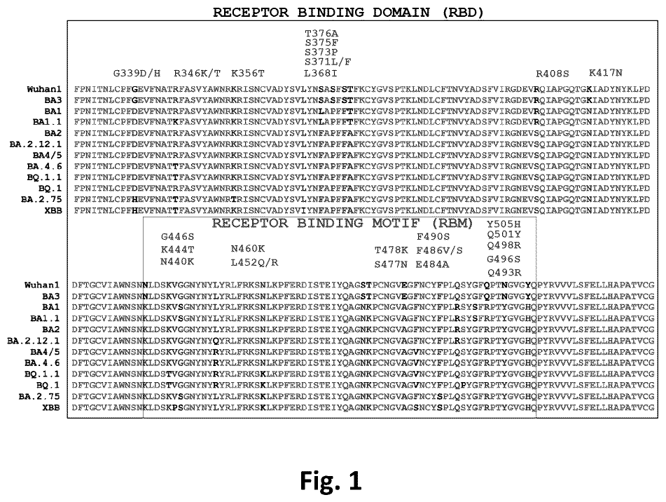

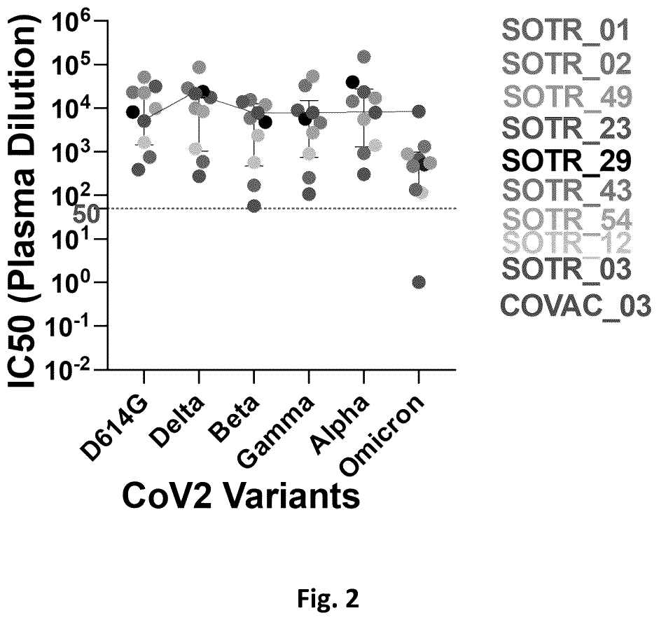

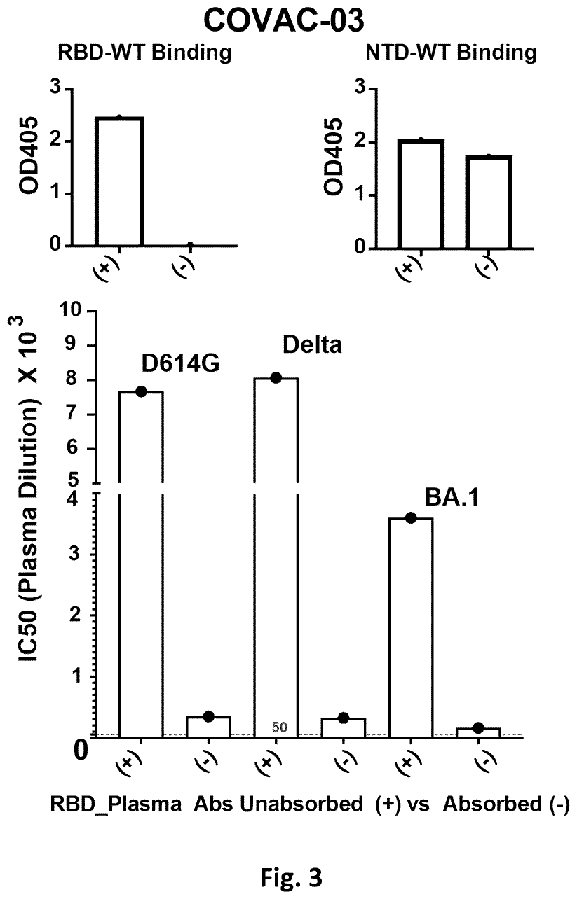

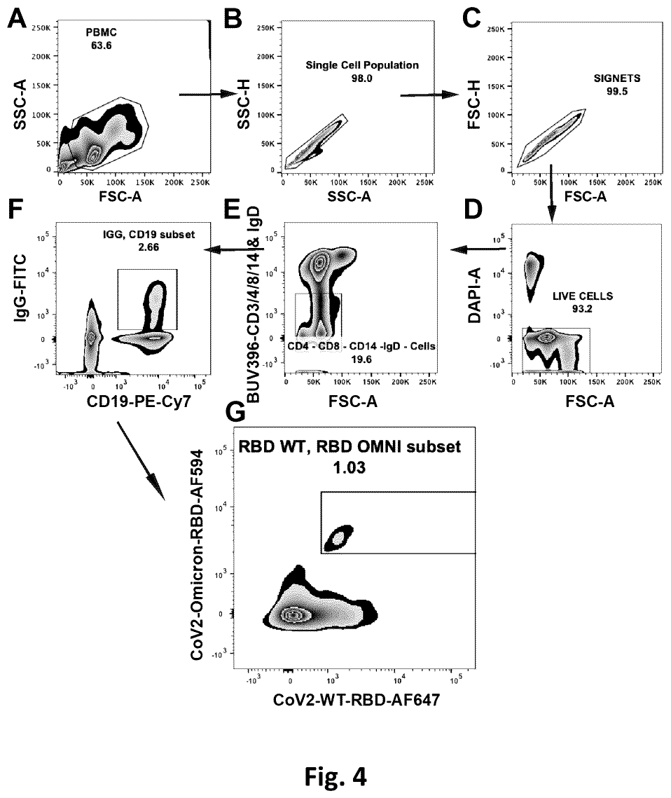

The emergence of the highly mutated Omicron (B.1.1.529) variant led to a significant reduction in COVID-19 vaccine plasma neutralization potency as compared to their efficacy against alpha (B.1.1.7), beta (B.1.351.1), gamma (P.1), and delta (B.1.617.2) variants. Comparison of RBD amino acid sequences from different CoV2 variants and Omicron subvariants demonstrates the extensive mutations in the Omicron strains that are responsible for the complete loss or reduced neutralization potency of different therapeutic monoclonal antibodies (FIG. 1). The presence of cross-CoV2 neutralizing plasma antibodies was demonstrated in CoV2 infected/COVID-19 vaccinated subjects. Similar to earlier observations, a significant decrease in IC50 was observed against Omicron as compared to other SARS-CoV2 variants for all the plasma except one, COVAC-03, which had comparable IC50 against all the variants, including Omicron (FIG. 2). Notably, COVAC-03 was the only subject reported with BA.1 infection while the timing of the infections indicated that all other subjects had either B.1.1.7 or B.1.617.2 infections. To determine the fraction of the broadly neutralizing antibodies present in COVAC-03 plasma that were RBD-directed, the RBD-specific antibodies in this plasma were absorbed with RBD WT recombinant protein bound to Sepharose beads. This resulted in a >90% decrease in neutralization activity against both CoV2WT and BA.1, which indicated that broadly conserved epitopes in the RBD were the targets of the dominant pan-CoV2 neutralizing antibodies in COVAC03 plasma (FIG. 3). Peripheral blood mononuclear cells (PBMC) from COVAC03 were used to sort RBD^{WT+} and RBD^{BA.1+}-bound double-antigen-positive IgG-expressing memory B cells sorting with a strategy shown in FIG. 4.

Conserved Neutralizing Epitope on RBD is not Immunodominant.

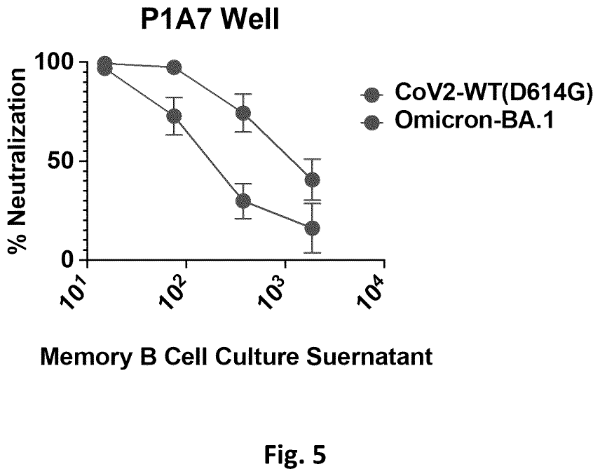

One of 288 wells with sorted single memory B cells showed cross-CoV2 neutralization. A total of two hundred eighty-eight RBDWT+ RBDBA.1+ double-positive memory B cells were sorted into three different 96 wells flat bottom culture plates pre-plated with MS40L cells and supplemented with FBS, cytokines (IL2, IL4, IL10, IL21), and CpG in AMEM and incubated in a 5% CO2 incubator for B cell proliferation, as described previously (Choudhary A., et al. J Immunol. 2018; 200(9):3053-3066). An average of 17-18 cycles of cell division were observed in two weeks where a single cell divided to reach an average of 125,000 cells (this proliferation rate compared to two B cell divisions per LZ-DZ cycle in the germinal center in approximately 24 h). After 14 days, the B cell culture supernatants were screened for RBD_{WT} and RBD_{BA.1} binding antibodies by ELISA. A total of 45 RBD IgG-positive wells were detected; 28 showed binding with RBD-WT and RBDBA.1, 16 bound only with RBD-wt, and one well showed weak binding to RBDBA.1. The majority of the wells contained antibodies that were non-neutralizing (NN), three wells showed weak neutralization against BA.1 but not against CoV2^{WT}, and only one out of the 28 RBDWT+BA.1+ double-positive well supernatants (plate 1, well A7) possessed potent neutralizing activity against both CoV2-wt and BA.1 pseudoviruses (FIG. 5). Heavy and Light chain variable regions were cloned from this well and cloned in IgG1 H and L chain vectors to generate a functional antibody, called Acovimab.

Recombinant ACOVIMAB, a Potent Cross-CoV2-Neutralizing Human Monoclonal Antibody, Belongs to the VH1-58 Class.

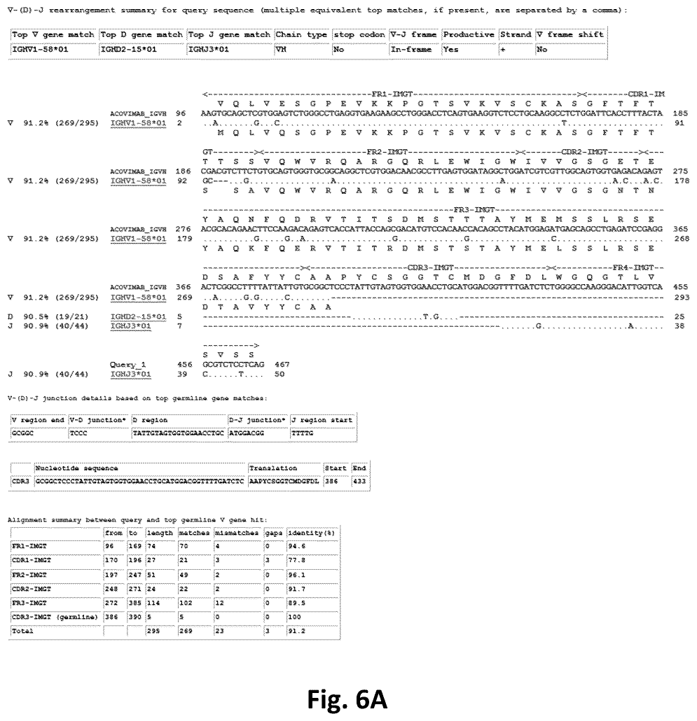

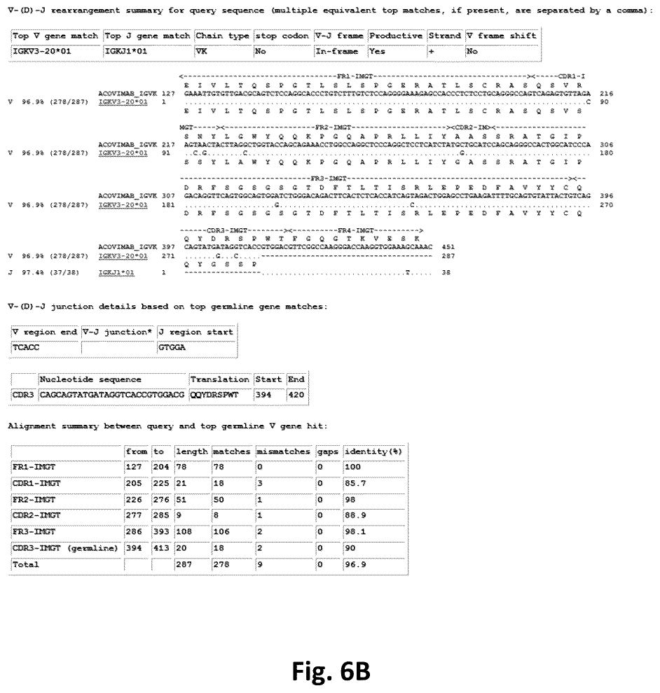

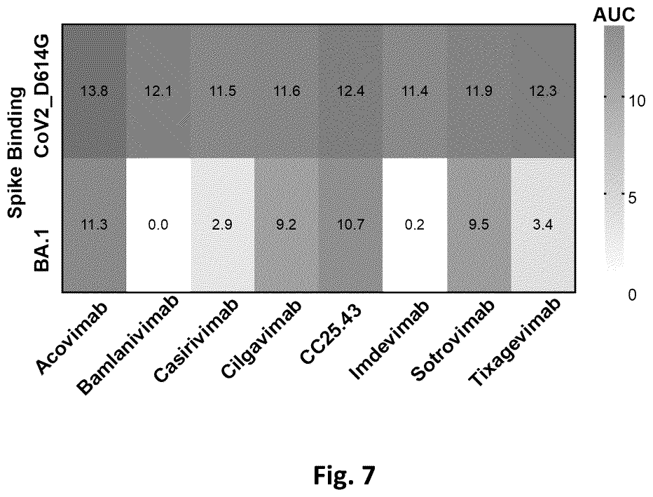

Genetic analysis of recombinant Acovimab demonstrated the use of IGVH1-58 and IGVK3-20 genes; the use of these germline sequences was previously reported for a number of CoV2 neutralizing antibodies, including AZD8895, COV2-2196, Tixagevimab, REGN10933, Casirivimab, and BD-836. However, none of the previously described VH1-58/VK3-20 mAbs showed a high level of somatic hypermutations, whereas Acovimab IGVH possessed a 15.3% mutation rate and the insertion of one amino acid in VH-CDR1 compared to the germline IGVH1-58 amino acid sequence, while the Acovimab light chain possessed a moderate level of mutation (4.5%) in amino acid sequence compared to its IGVK3-20*01 germline sequence (FIGS. 6A-B). Unlike the previously reported mAbs of the IGVH1-58 class, Acovimab showed significantly strong binding (FIG. 7) and neutralization breadth against many Omicron subvariants (BA.1, BA.2, BA.2.12.1, BA.2.75, BA4/5, and BA.4.6), and was relatively weakly affected by the F486V substitution present in BA4/5 (IC50=0.28 ug/ml) and the R346T substitution present in BA.4.6 (IC50=0.15 ug/ml). Acovimab possessed very high neutralization potency for many of the Omicron subvariants, including BA.1, BA.2, 2.12.1, and BA.2.75, (IC50 of 1-3 ng/ml); this was much stronger than the IC50 value of 136 ng/ml previously reported for the most potent IGVH1-58 class of CoV2 mAb for the Omicron_{BA.1} variant (Table 3). However, the neutralization potency of Acovimab was lower against the BA.4/5 and BA.4.6 variants (IC50 of 150 ng/ml), which contained the R346T and F486V substitutions. Of note, the residual neutralization potency of Acovimab for BA.4.6 contrasted with the complete loss of sensitivity of this variant to Evusheld (a mixture of Tixagevimab+Cilgavimab), which has EUA approval for pre-exposure prophylaxis of COVID-19 in immunocompromised individuals.

TABLE 3

| MAb (Name) | VDJ % Match (amino acid) | | IC50 vs BA1 ng/ml | pM |
|---|---|---|---|---|
| ACOVIMAB_IgG | 124/124 | | 0.9 | 60 |
| CV2.1169_IgA | 108/124 | (87%) | 136 | 850 |
| CS44 | 105/124 | (84.7%) | >15.000 | >100,000 |
| AZD8895 (TIXAGEVIMAB) | 102/124 | (82%) | 600 | 4,000 |
| UT28K | 101/124 | (81%) | 750 | 5,000 |

### Example 3

This Example describes the materials and methods for Example 4.

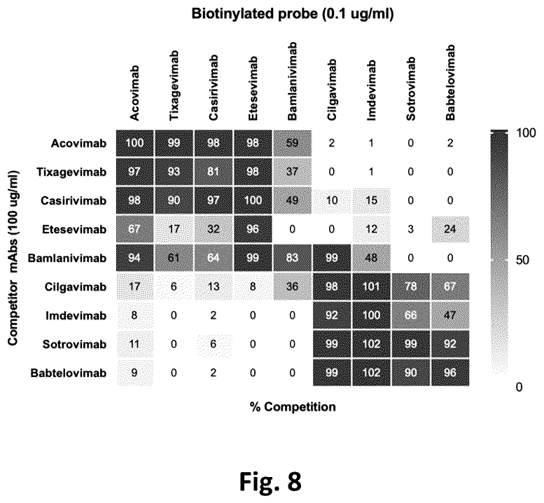

Anti-RBD mAbs binding competition to RBD-B.1 To identify the overlapping epitope of RBD targeting antibodies, the mAbs competition assay was first performed to bind with RBD-B.1 (SARS-CoV2-WT). To achieve this, all mAbs were biotinylated using standard protocol. In brief, 100 μg of mAb was diluted in 80 μl Milli-Q-H2O followed by addition of 10 μl of 1 M NaHCO₃ and 10 μg of biotin (10 mg/ml in DMSO) N-hydroxysuccinimiso-biotin (Sigma #H-1759). Mixed well and incubated for 4 hours at RT followed by 0/N at 4° C. Added 10 μl of 1 M glycine to block the free biotin and incubated for a minimum of 30 min at RT before use. Optionally spin column was used to remove the free biotin. For the RBD binding competition each biotinylated mAbs (100 ng/ml) were competed by unbiotinylated anti-RBD-mAbs including unbiotinylated version of itself (100 μg/ml) and % inhibition were plotted as heat map to separate them into major groups based on their binding competition pattern (FIG. 8).

SARS-CoV2 neutralization potency of anti-RBD mAbs. CoV2-psV (pseudovirion) were generated by transient transfection of HEK293T cells with Codon-optimized Wuhan-Hu-1, B.1 D614G, B.1.1.7 or alpha, B.1.351 or beta, P.1 or gamma, B.617.2 or delta, and omicron subvariants (BA.1, BA.2, BA.2.12.1, BA.2.75, BA.4/5, BA.4.6, BQ.1.1, and XBB.1.5) spike gene sequences with 18 aa C-terminal truncations were cloned into the pCDNA3.1vector. Six of the earlier CoV2 variants and eight different Omicron subvariants spike pseudotyped retroviral pseudovirus particles were generated by co-transfecting spike and pnl4.3. Luc.r⁻e⁻ plasmids into HEK 293T cells to produce the CoV2 psV and HuACE2-HeLa cells were infected with the CoV2 psV in DMEM media supplemented with polybrene (10 μg/ml), and infectivity was determined by reading luciferase activity in the cell after 72 h post-infection upon adding luciferase substrate (Britelite, PE) to the cell-lysate. To determine the neutralization potency of mAbs, psV dilutions, 100,000 RLU was used to infect Hu-ACE2-HeLA cells in the presence of titrated antibodies. The neutralization potency of mAbs, defined as the mAb concentration, required to reduce viral infection by 50% (IC50), was calculated using One-Site Fit Log IC₅₀ regression (Table 4).

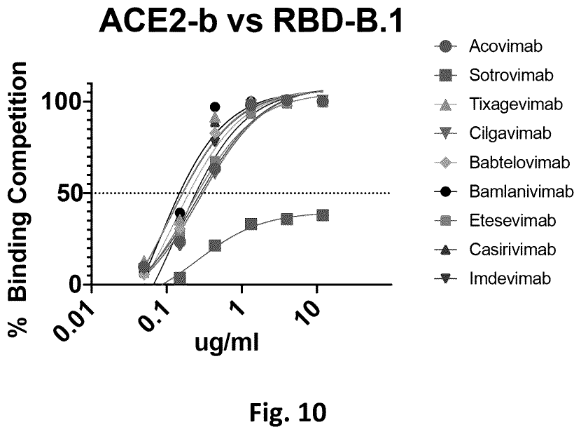

Anti-RBD mAbs binding competition with soluble ACE2 to bind with RBD-B.1 To identify if the anti-RBD-mAbs neutralize the SARS-CoV2 by blocking the RBD-ACE2 binding, the mAbs competition assay was performed to compete with soluble biotinylated-ACE2 for binding with RBD-B.1 (SARS-CoV2-WT) coated on the ELISA plate. To achieve this all mAbs were tested in duplicate in titration to compete with 100 ng/ml biotinylated-ACE2 for binding with RBD, and % inhibition was plotted (FIG. 10).

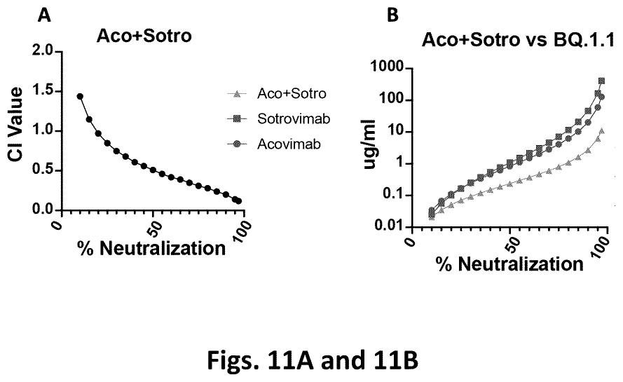

Synergy Calculation The neutralization of BQ.1.1 with Acovimab, Sotrovimab, and the combination of the two mAbs in a 1:1 ratio was determined by titrating the antibody 4-fold, starting from 50 μg/ml. To assess the allosteric effect of one mAb when the other is already present, 50 μg/ml of Acovimab was added to 50 ug/ml of Sotrovimab. The percentage of neutralization was calculated for each concentration of the mAb, and the data was entered into the CompySyn software (Chou T C, et al. Adv Enzyme Regul. 1984; 22:27-55). This is an automated data analysis program based on a mass-action law equations/combination index (CI) algorithm that quantitates synergism or antagonism in drug combinations. The software produces different plots, including the % Neutralization-CI plot (where CI<1 is synergism and CI>1 is antagonism (FIG. 11A), and the % Neutralization-DRI (Drug Reduction Index) plot that provides a quantitative value of synergism or antagonism (FIG. 11B).

### Example 4

Two independent motifs or immunogenic epitopes were identified on SARS-CoV2 spike, which can be targeted together by two different antibodies or other biomolecules to have a strong synergistic effect as virus entry inhibitors. Discontinuous epitopes identified for Sotrovimab binding flanking S: T331 to C361 outside the receptor binding motif (RBM) in receptor binding domain (RBD) of Acovimab are focused in the RBM in the region S: L455 to Q493.

Acovimab is a Class I Anti-RBD mAb and Neutralizes the Virus by Blocking the RBD-ACE2 Binding.

A total of nine anti-RBD mAbs, including Acovimab and eight well-characterized anti-RBD mAbs that have been previously approved by FDA for therapeutic use, were tested in RBD-B.1 binding competition assays, to identify shared binding competition patterns. Published cryo-EM structural data have shown that the eight therapeutically approved RBD mAbs fall into three different classes. Tixagevimab, Casirivimab, and Etesevimab are class 1 mAbs, which only bind to the RBD on Spike in its up form. Bamlanivimab is a class 2 RBD mAb, that binds to the RBD on the Spike in its down form. Cilgavimab, Imdevimab, Sotrovimab, and Bebtelovimab are class 3 RBD mAbs, which can bind to the RBD in both its up and down forms (Barnes C O, et al. Nature. 2020; 588(7839):682-687; Chen Y, et al. Nat Rev Immunol. 2022:1-11.).

These assignments were consistent with the binding competition patterns observed (FIG. 8). The three class 1 mAbs cross-competed with each other and with Acovimab, showing that Acovimab also belonged to the class 1 group. The four class 3 mAbs also efficiently cross-competed with each other, but not with any of the class 1 mAbs. Bamlanivimab, the only class 2 mAb, had a unique competition pattern, as it bridged some of class 1 and class 3 sites. In addition to competing with all 4 class 1 mAbs, Bamlanivimab also competed with two of the class 3 mAbs (strongly with Cilgavimab and weakly with Imdevimab), but not with either Sotrovimab or Babtelovimab, the other two class 3 mAbs. Notably, the competition between Bamlanivimab and Etesevimab was not reciprocal; although Bamlanivimab strongly competed (99% competition) with binding of labeled Etesevimab to the wt RBD, Etesevimab did not block any of the binding of Bamlanivimab (0% competition).

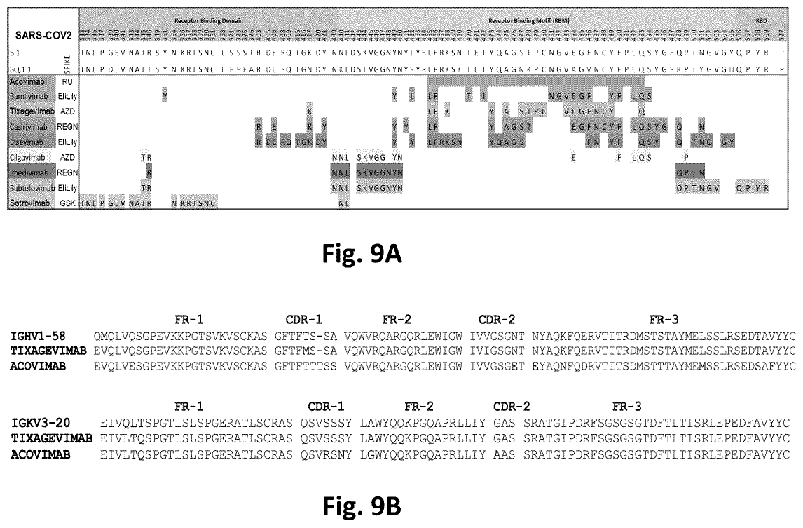

The footprints of all eight mAbs derived from the cryo-EM structures are shown in FIG. 9A, and the sequence homology of Acovimab and Tixagevimab with the germline VH sequence is shown in FIG. 9B. The class and binding region of Acovimab was predicted on the basis of its competition with the other class 1 mAbs and its sequence homology with Tixagevimab.

A Combination of Acovimab and Sotrovimab Neutralize the SARS-CoV2 Omicron Subvariant BQ.1.1 Synergistically.

The neutralization activity of Acovimab and the other mAbs described above for a panel of 13 CoV2 sequences are summarized in Table 4 as IC50. Evusheld is a combination of class I and class III mAbs Tixagevimab and Cilgavimab. These two mAbs have been shown earlier to synergistically neutralize the CoV2 variants. However, Evusheld lost effectiveness against subvariants of Omicron which emerged later, i.e., BA.4.6, BQ.1.1, and XBB.1.5 (Table 4).

TABLE 4
IC50 values of anti-RBD mAbs including Acovimab against 14 SARS-CoV2 variants
performed with HuACE2-HeLa cells based pseudovirus neutralization assay.

| | | IC50 (ug/ml) | | IC50 (ug/ml) | | | | | | |
|---|---|---|---|---|---|---|---|---|---|---|
| | CoV2 | RU | GSK | Evusheld Eli Lily Regeneron (AstraZeneca) | | | | | | |
| CoV2 | Pango | Aco- | Sotro- | Tixage- | Cilga- | Bebtelo- | Etese- | Bamlani- | Casiri- | Imde- |
| Variant Name | Lineage | vimab | vimab | vimab | vimab | vimab | vimab | vimab | vimab | vimab |
| Wuhan_Hu-1 | B.1_614D | 0.0090 | 0.070 | 0.0032 | 0.011 | 0.0028 | 0.012 | 0.0053 | 0.0062 | 0.0048 |
| Wuhan_Hu-1_D614G | B.1 | 0.0059 | 0.033 | 0.0012 | 0.0069 | 0.0019 | 0.0066 | 0.0020 | 0.0023 | 0.0025 |
| Alpha | B.1.1.7 | 0.0052 | 0.094 | 0.0017 | 0.010 | 0.0020 | 0.10 | 0.0034 | 0.0052 | 0.0028 |
| Delta | B.1.617.2 | 0.0051 | 0.13 | 0.0008 | 0.018 | 0.0015 | 0.0058 | 0.59 | 0.0027 | 0.0035 |
| Beta | B.1.351 | 0.0020 | 0.011 | 0.0054 | 0.003 | 0.0009 | >5 | 0.21 | 0.094 | 0.0028 |
| Gamma | P.1 | 0.0029 | 0.058 | 0.0060 | 0.005 | 0.0017 | >5 | 1.4 | 0.18 | 0.0019 |
| | BA.1 | 0.0013 | 0.18 | 0.14 | 0.21 | 0.0023 | >5 | >5 | >5 | >5 |
| Omicron | BA.2 | 0.0029 | 0.15 | >5 | 0.0091 | 0.0018 | >5 | >5 | >5 | 0.51 |
| sub- | BA.2.12.1 | 0.0037 | 1.5 | 0.56 | 0.019 | 0.0023 | >5 | >5 | >5 | 0.10 |
| variants | | | | | | | | | | |
| | BA.2.75 | 0.0037 | 0.027 | 0.11 | 0.055 | 0.0033 | >5 | >5 | 0.44 | >5 |
| | BA.4/5 | 0.22 | 0.47 | >5 | 0.018 | 0.0014 | >5 | >5 | >5 | 0.32 |
| | BA.4.6 | 0.22 | 2.5 | >5 | >5 | 0.0013 | >5 | >5 | >5 | 0.18 |
| | BQ.1.1 | 0.29 | 0.69 | >5 | >5 | >5 | >5 | >5 | >5 | >5 |

Acovimab and Sotrovimab do not compete with each other for binding to the RBD, and have comparable effectiveness in neutralizing the Omicron subvariant BQ.1.1 virus. Acovimab binds to key residues in the RBM and neutralizes the virus by competing with ACE2 for binding to the RBD. Sotrovimab binds outside of the RBM, and its virus-neutralizing mechanism is independent of ACE competition (FIG. 10). Based on these properties, it was hypothesized that the combination of Acovimab and Sotrovimab could act synergistically to neutralize the highly resistant BQ.1.1 virus.

Synergy is defined as an interaction of elements that produces an effect that is greater than the effect that would have resulted from simply adding up the effects of each individual element. Thus, if two mAbs neutralize synergistically, this would mean that the degree of neutralization achieved by a combination of the two agents at a given concentration would be greater than the effect of either of the antibodies at the same concentration. The advantage of this is that a lower effective total antibody concentration would be required to achieve sterilizing immunity, and that a given total dose of the antibodies would therefore remain effective for a longer period.

A standard method for examining whether a combination of two therapeutics work synergistically with each other is to calculate their combination index (CI) (Chou T C, et al. Adv Enzyme Regul. 1984; 22:27-55; Zhang N, et al. Am J Cancer Res. 2016; 6(1):97-104.). This is defined by the following formula: CI (at a given % neutralization)=(D)₁/(D_{X})₁+(D)₂/(D_{X})₂+α(D)₁(D₂)/(D_{X})₁/(D_{X})₂, where (D₁) and (D₂) are the concentrations of HuMAbs 1 and 2, respectively, in the mixture used to achieve a given percent neutralization, and (D_{X})₁ and (D_{X})₂ are the concentrations of HuMAbs 1 and 2, respectively, used alone to achieve that same percent neutralization. For HuMAbs having different modes of action or acting independently (mutually nonexclusive), α=1. For HuMAbs having the same or similar modes of action (mutually exclusive), α=0 (Chou T C. Cancer Res. 2010; 70(2):440-446.). CI values are routinely determined using the CompuSyn software program (Chou T C, et al. Adv Enzyme Regul. 1984; 22:27-55), and CI values of less than 1 indicate synergy, while values above 1 reflect antagonism.

The CompuSyn software program was used to calculate the quantitative value of the synergy between Acovimab and Sotrovimab for the BQ.1.1 pseudotype virus. The calculated neutralization values for Acovimab, Sotrovimab, and their combination in a 1:1 ratio are shown in Table 5, and the graphs showing levels of CI and % neutralization at different antibody doses are shown in FIGS. 11A-B.

TABLE 5
Neutralization potency of the antibodies.

| % | | | | | | |
|---|---|---|---|---|---|---|
| Neutral- | Aco- | Sotro- | Aco + | | | CI |
| ization^{€} | vimab | vimab | Sotro | DRI_Aco | DRI_Sotro | Value |
| 10 | 0.03 | 0.03 | 0.02 | 1.65 | 1.20 | 1.44 |
| 15 | 0.07 | 0.06 | 0.04 | 1.93 | 1.59 | 1.15 |
| 20 | 0.11 | 0.10 | 0.05 | 2.17 | 1.96 | 0.97 |
| 25 | 0.17 | 0.17 | 0.07 | 2.39 | 2.33 | 0.85 |
| 30 | 0.24 | 0.25 | 0.09 | 2.61 | 2.71 | 0.75 |
| 35 | 0.34 | 0.38 | 0.12 | 2.82 | 3.11 | 0.68 |
| 40 | 0.46 | 0.54 | 0.15 | 3.03 | 3.54 | 0.61 |
| 45 | 0.62 | 0.77 | 0.19 | 3.25 | 4.01 | 0.56 |
| 50 | 0.83 | 1.08 | 0.24 | 3.48 | 4.52 | 0.51 |
| 55 | 1.11 | 1.52 | 0.30 | 3.72 | 5.10 | 0.46 |
| 60 | 1.50 | 2.16 | 0.37 | 3.99 | 5.77 | 0.42 |
| 65 | 2.04 | 3.12 | 0.47 | 4.29 | 6.56 | 0.39 |
| 70 | 2.84 | 4.60 | 0.61 | 4.64 | 7.53 | 0.35 |
| 75 | 4.08 | 7.07 | 0.81 | 5.05 | 8.76 | 0.31 |
| 80 | 6.19 | 11.56 | 1.11 | 5.57 | 10.41 | 0.28 |
| 85 | 10.25 | 20.97 | 1.63 | 6.28 | 12.84 | 0.24 |
| 90 | 20.02 | 46.25 | 2.73 | 7.34 | 16.97 | 0.20 |
| 95 | 59.03 | 165.86 | 6.23 | 9.47 | 26.61 | 0.14 |
| 97 | 127.44 | 411.54 | 11.23 | 11.35 | 36.64 | 0.12 |

^{€}SARS-CoV2 Omicron subvariant BQ.1.1 neutralization was done with Acovimab, Sotrovimab, and with their combination in 1:1 ratio. The neutralization potency of mAbs, defined as the mAb concentration, required to reduce viral infection by % is calculated using One-Site Fit LogIC₅₀ regression. Combination Index (CI) and Dose Reduction Index were calculated using CompuSyn software. CI < 1 indicate synergism.

The results revealed substantial synergy between Acovimab and Sotrovimab in neutralization of BQ.1.1, with the synergy increasing as neutralization levels increased (CI: 0.97 at 20%, CI: 0.51 at 50%, and CI: 0.11 at 97% neutralization) (FIG. 11A). This translated to an impressive reduction in total antibody dose required to achieve near sterilizing levels of protection. When used individually, the concentrations of Acovimab and Sotrovimab required to achieve 97% neutralization (IC₉₇ values) were 127 μg/mL and 411 μg/mL, respectively, while the same level of neutralization required a combined mAb dose of only 11 ug/mL (Table 5 and FIG. 11B). This translated into a >11-fold dose reduction of Acovimab and >36-fold reduction of Sotrovimab needed to achieve a 97% level of virus neutralization by the combination. This increased potency should dramatically increase the effectiveness of this therapy in vivo, and also significantly increase the length of time the treatment would remain effective.

A Chimeric CoV1-CoV2 RBD Antigen Design for a Focused and Synergistic Immunoprotective Response.

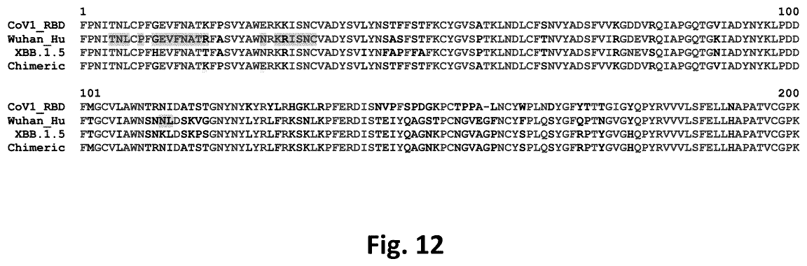

Sotrovimab mAb was isolated from a CoV-1 infected individual and neutralized the CoV1 more efficiently than CoV2. This indicates a better presentation of the Sotrovimab epitope in CoV1 than CoV2. However, it neutralizes all of the CoV2 subvariants with different potencies and shows synergy with CoV2 RBM-directed antibodies like Acovimab. It was thus hypothesized that designing a chimeric spike with a CoV1 sequence presenting the Sotrovimab epitope in the CoV2-Omicron subvariant XBB.1.5 spike backbone (FIG. 12). It is advantageous to possess both the native Sotrovimab together with the native XBB.1.5 RBM, as it likely results in more efficient induction of Sotrovimab-like antibodies after immunization, as well as XBB.1.5-specific anti-RBM antibodies, which may also neutralize synergistically.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. An isolated anti-SARS-CoV-2 antibody or antigen-binding fragment thereof that binds specifically to a SARS-CoV-2 antigen, wherein the antibody or antigen-binding fragment thereof comprises: three heavy chain complementarity determining regions (HCDRs) (HCDR1, HCDR2, and HCDR3) of a heavy chain variable region (HCVR) having the amino acid sequence of SEQ ID NO: 7; and three light chain CDRs (LCDR1, LCDR2, and LCDR3) of a light chain variable region (LCVR) having the amino acid sequence of SEQ ID NO: 8.

2. The antibody or antigen-binding fragment thereof of claim 1, wherein HCDR1, HCDR2, HCDR3, LCDR1, LCDR2, and LCDR3 comprise respective amino acid sequences of SEQ ID NOs: 1-6.

3. The antibody or antigen-binding fragment thereof of claim 2, comprising a HCVR having the amino acid sequence of SEQ ID NO: 7 and/or comprising a LCVR having the amino acid sequence of SEQ ID NO: 8.

4. The antibody or antigen-binding fragment thereof of claim 2, comprising a heavy chain having the amino acid sequence of SEQ ID NO: 9 and/or comprising a light chain having the amino acid sequence of SEQ ID NO: 10.

5. A pharmaceutical composition comprising a first isolated anti-SARS-CoV-2 antibody or antigen-binding fragment thereof according to claim 1, and a second isolated anti-SARS-CoV-2 antibody or antigen-binding fragment thereof that binds specifically to the SARS-CoV-2 antigen, wherein the first anti-SARS-CoV-2 antibody or antigen-binding fragment thereof and the second anti-SARS-CoV-2 antibody or antigen-binding fragment thereof bind to different epitopes of the SARS-CoV-2 antigen and a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

6. The pharmaceutical composition of claim 5, wherein the second anti-SARS-CoV-2 antibody comprises HCDR1, HCDR2, HCDR3, LCDR1, LCDR2, and LCDR3 of Sotrovimab having respective amino acid sequences of SEQ ID NOs: 37-42.

7. The pharmaceutical composition of claim 6, wherein the second anti-SARS-CoV-2 antibody comprises HCVR of Sotrovimab having the amino acid sequence of SEQ ID NO: 43 and/or LCVR of Sotrovimab having the amino acid sequence of SEQ ID NO: 44.

8. The pharmaceutical composition of claim 6, wherein the second anti-SARS-CoV-2 antibody comprises Sotrovimab.

9. A method of neutralizing SARS-CoV-2 or treating a SARS-CoV-2 infection in a subject, comprising administering to a subject in need thereof a therapeutically effective amount of a first isolated anti-SARS-CoV-2 antibody or antigen-binding fragment thereof according to claim 1, and a therapeutically effective amount of a second isolated anti-SARS-CoV-2 antibody or antigen-binding fragment thereof that binds specifically to the SARS-CoV-2 antigen, wherein the first anti-SARS-CoV-2 antibody or antigen-binding fragment thereof and the second anti-SARS-CoV-2 antibody or antigen-binding fragment thereof bind to different epitopes of the SARS-CoV-2 antigen and a combination of the first anti-SARS-CoV-2 antibody or antigen-binding fragment thereof and the second anti-SARS-CoV-2 antibody or antigen-binding fragment thereof exhibits a synergistic effect in neutralizing SARS-CoV-2 variants.

10. The method of claim 9, wherein the second anti-SARS-CoV-2 antibody comprises HCDR1, HCDR2, HCDR3, LCDR1, LCDR2, and LCDR3 of Sotrovimab having respective amino acid sequences of SEQ ID NOs: 37-42.

11. The method of claim 10, wherein the second anti-SARS-CoV-2 antibody comprises HCVR of Sotrovimab having the amino acid sequence of SEQ ID NO: 43 and/or LCVR of Sotrovimab having the amino acid sequence of SEQ ID NO: 44.

12. The method of claim 11, wherein the second anti-SARS-CoV-2 antibody comprises Sotrovimab.

13. An isolated polypeptide comprising an amino acid sequence having at least 75% sequence identity to the amino acid sequence of SEQ ID NO: 13 or having the amino acid sequence of SEQ ID NO: 13.

14. A polynucleotide encoding the polypeptide of claim 13.

15. A vector comprising the polynucleotide of claim 14.

16. A host cell comprising the polynucleotide of claim 14.

17. A virus-like particle comprising the polynucleotide of claim 14.

18. An immunogenic composition for stimulating an immune response in a subject in need thereof, comprising the polynucleotide of claim 14.

19. The immunogenic composition of claim 18, which is capable of inducing production of at least two anti-SARS-CoV2 antibodies that bind specifically to different epitopes on a spike protein of a SARS-CoV2 variant.

20. A method of stimulating an immune response, neutralizing SARS-CoV-2, or treating a SARS-CoV-2 infection in a subject in need thereof, comprising administrating to the subject an effective amount of the polynucleotide of claim 14.
